(19) United States
(12) Patent Application Publication
(10) Pub. No.: US 2022/0378528 A1
(43) Pub. Date: Dec. 1, 2022

(54) SYSTEMS AND METHODS FOR CONTROLLING A SURGICAL ROBOTIC ASSEMBLY IN AN INTERNAL BODY CAVITY

(71) Applicant: Vicarious Surgical Inc., Waltham, MA (US)

(72) Inventors: Sammy Khalifa, Medford, MA (US); Adam Sachs, Somerville, MA (US); Michael Cattafe, Concord, MA (US); Theodore Aronson, Somerville, MA (US); Zachary Deocadiz-Smith, Auburndale, MA (US)

(21) Appl. No.: 17/826,120

(22) Filed: May 26, 2022

Related U.S. Application Data
(60) Provisional application No. 63/193,296, filed on May 26, 2021.

(51) Int. Cl. A61B 34/30; A61B 90/00; A61B 34/00

(52) U.S. Cl. CPC A61B 34/30; A61B 90/361; A61B 34/70; A61B 2034/305

(57) ABSTRACT

Methods and systems for performing a surgery within an internal cavity of a subject are provided herein. An example method for controlling a robotic assembly of a surgical robotic system includes, while at least a portion of the robotic assembly is disposed in an interior cavity of a subject, receiving a first control mode selection input from an operator and changing a current control mode of the surgical robotic system to a first control mode in response to the first control mode selection input; while the surgical robotic system is in the first control mode, receiving a first control input from hand controllers; in response to receiving the first control input, changing a position and/or an orientation of: at least a portion of the camera assembly, of at least a portion of the robotic arm assembly, or both, while maintaining a stationary position of instrument tips of the end effectors disposed at distal ends of the robotic arms.

### RELATED APPLICATIONS

This application claims priority to U.S. Provisional Application No. 63/193,296 filed on May 26, 2021, the entire content of which is hereby incorporated by reference in its entirety.

### BACKGROUND OF THE INVENTION

Since its inception in the early 1990s, the field of minimally invasive surgery has rapidly grown. While minimally invasive surgery vastly improves patient outcome, this improvement comes at a cost to the surgeon's ability to operate with precision and ease. During conventional laparoscopic procedures, the surgeon typically inserts a laparoscopic instrument through multiple small incisions in the patient's abdominal wall. The nature of tool insertion through the abdominal wall constrains the motion of the laparoscopic instruments, as the instruments are unable to move side-to-side without injury to the abdominal wall. Standard laparoscopic instruments are also limited in motion, and are typically limited to four axes of motion. These four axes of motion are movement of the instrument in and out of the trocar (axis 1), rotation of the instrument within the trocar (axis 2), and angular movement of the trocar in two planes while maintaining the pivot point of the trocar's entry into the abdominal cavity (axes 3 and 4). For over two decades, the majority of minimally invasive surgery has been performed with only these four degrees of motion. Moreover, prior systems require multiple incisions if the surgery requires addressing multiple different locations within the abdominal cavity.

Existing robotic surgical devices attempted to solve many of these problems. Some existing robotic surgical devices replicate non-robotic laparoscopic surgery with additional degrees of freedom at the end of the instrument. However, even with many costly changes to the surgical procedure, existing robotic surgical devices have failed to provide improved patient outcome in the majority of procedures for which they are used. Additionally, existing robotic devices create increased separation between the surgeon and surgical end-effectors. This increased separation can causes injuries resulting from the surgeon's misunderstanding of the motion and the force applied by the robotic device. Because the degrees of freedom of many existing robotic devices are unfamiliar to a human operator, surgeons need extensive training on robotic simulators before operating on a patient in order to minimize the likelihood of causing inadvertent injury.

To control existing robotic devices, a surgeon typically sits at a console and controls manipulators with his or her hands and/or feet. Additionally, robot cameras remain in a semi-fixed location, and are moved by a combined foot and hand motion from the surgeon. These semi-fixed cameras offer limited fields of view and often result in difficulty visualizing the operating field.

Other robotic devices have two robotic manipulators inserted through a single incision. These devices reduce the number of incisions required to a single incision, often in the umbilicus. However, existing single-incision robotic devices have significant shortcomings stemming from their actuator design. Existing single-incision robotic devices include servomotors, encoders, gearboxes, and all other actuation devices within the in vivo robot, which results in relatively large robotic units that are inserted within the patient. This size severely constrains the robotic unit in terms of movement and ability to perform various procedures. Further, such a large robot typically needs to be inserted through a large incision site, oftentimes near the size of open surgery, thus increasing risk of infection, pain, and general morbidity.

A further drawback of conventional robotic devices is their limited degrees of freedom of movement. Hence, if the surgical procedure requires surgery at multiple different locations, then multiple incision points need to be made to be able to insert the robotic unit at the different operating locations. This increases the chance of infection of the patient.

### SUMMARY OF THE INVENTION

The present disclosure provides methods for controlling a robotic assembly of a surgical robotic system when at least a portion of the robotic assembly is disposed in an interior cavity of a subject. The robotic assembly includes a camera assembly and a robotic arm assembly including a first robotic arm and a second robotic arm defining a virtual chest of the robotic arm assembly. Some methods include changing a control mode of the surgical robotic system from a current control mode to a control mode in which a position and/or an orientation of a virtual chest of the robotic arm assembly is changed using motion of hand controllers while end effectors of the robotic arms remain stationary. Some methods include changing a control mode of the surgical robotic system from a current control mode to a control mode in which a direction of view of the camera assembly is changed using hand controllers while the instrument tips of the end effectors of the robotic arms of the arm assembly remain stationary. The present disclosure also provides surgical robotic systems providing a plurality of control modes including one or more of the aforementioned control modes and/or other control modes described herein. The present disclosure also provides computer readable media that, when executed on one or more processors of a computing unit of a surgical robotic system, provide one or more control modes described herein, and/or execute any of the methods described herein.

In a first aspect, the present invention provides a method for controlling a robotic assembly of a surgical robotic system. The surgical robotic system includes an image display, hand controllers configured to sense a movement of an operator's hands, and the robotic assembly. The robotic assembly includes a camera assembly and a robotic arm assembly including a first robotic arm and a second robotic arm. The method includes, while at least a portion of the robotic assembly is disposed in an interior cavity of a subject, receiving a first control mode selection input from the operator and changing a current control mode of the surgical robotic system to a first control mode in response to the first control mode selection input. The method further includes, while the surgical robotic system is in the first control mode, receiving a first control input from hand controllers. The method further includes, in response to receiving the first control input, changing a position and/or an orientation of: at least a portion of the camera assembly, of at least a portion of the robotic arm assembly, or both, while maintaining a stationary position of instrument tips of end effectors disposed at distal ends of the robotic arms.

In one embodiment, the first robotic arm and the second robotic arm define a virtual chest of the robotic assembly, the virtual chest defined by a chest plane extending between a first pivot point of a most proximal joint of the first robotic arm, a second pivot point of a most proximal joint of the second robotic arm, and camera imaging center point of the camera assembly. A pivot center of the virtual chest lies midway along a line segment in the chest plane connecting the first pivot point of the first robotic arm and the second pivot point of the second robotic arm.

In one embodiment, the first control mode is a travel arm control mode or a camera control mode. Where the first control mode is a camera control mode, in response to receiving the first control input, the surgical robotic system changes an orientation and/or a positon of at least one camera of the camera assembly with respect to the current viewing direction while keeping the robotic arm assembly stationary. Where the first control mode is a travel arm control mode, in response to receiving the first control input, the surgical robotic system moves at least a portion of the robotic arm assembly to change a location of the virtual chest pivot center and/or an orientation the virtual chest with respect to the current viewing direction.

In one embodiment, the first control mode is a travel gestural arm control mode. The first control input corresponds to one of a plurality of gestural translation inputs or one of a plurality of gestural rotation inputs. Where the first control input corresponds to one of the plurality of gestural translation inputs, the surgical robotic system moves at least the portion of the robotic arm assembly to change the location of the virtual chest pivot center while maintaining the stationary position of the instrument tips of the end effectors in response to the first control input. Where the first control input corresponds to one of the plurality of gestural rotation inputs, the surgical robotic system moves at least the portion of the robotic arm assembly to change the orientation of the virtual chest with respect to the current viewing direction while maintaining the stationary position of the instrument tips of the end effectors.

In one embodiment, the plurality of gestural translation inputs include a pullback input in which the sensed movement of the hand controllers corresponds to the operator's hands moving back toward the operator's body, and where, when in the gestural arm control mode, the surgical robotic system moves at least the portion of the robotic arm assembly to move the location of the virtual chest pivot center forward in the current viewing direction in response to the pullback input. The plurality of gestural translation inputs further includes a push forward input in which the sensed movement of the hand controllers corresponds to operator's hands moving forward away from the operator's body, and where, when in the gestural arm control mode, the surgical robotic system moves at least the portion of the robotic arm assembly to move the location of the virtual chest pivot center back away from the current viewing direction in response to the push forward input.

In one embodiment, the plurality of gestural translation inputs comprises or further comprises a horizontal input, in which the sensed movement of the hand controllers corresponds to operator's hands moving in a horizontal direction with respect to the operator's body, and where, when in the gestural arm control mode, the surgical robotic system moves at least the portion of the robotic arm assembly to move the location of the virtual chest pivot center in a corresponding horizontal direction with respect to a current field of view of a current image displayed, and wherein the corresponding horizontal direction is a horizontal direction to the left or a horizontal direction to the right with respect to the current field of view of the current image displayed in response to the horizontal input.

In one embodiment, the plurality of gestural translation inputs comprises or further comprises a vertical input, in which the sensed movement of the hand controllers corresponds to operator's hands moving in a vertical direction with respect to the operator's body, and where, when in the gestural arm control mode, the surgical robotic system moves at least the portion of the robotic arm assembly to move the location of the virtual chest pivot center in a corresponding vertical direction with respect to a current field of view of a current image displayed, and wherein the corresponding vertical direction is a vertical up direction or a vertical down direction with respect to the current field of view of the current image displayed in response to the vertical input.

In one embodiment, the plurality of gestural rotation inputs comprises a right yaw input, in which a sensed movement of a left hand controller corresponds to a left hand of the operator moving forward away from the operator's body and a sensed movement of a right hand controller corresponds to a right hand of the operator moving back toward the operator's body, and where, when in the gestural arm control mode, the surgical robotic system moves at least the portion of the robotic arm assembly to yaw an orientation of the chest plane to the right about the virtual chest pivot center with respect to a current field of view of a current image displayed in response to the right yaw input, and a left yaw input, in which the sensed movement of the left hand controller corresponds to the operator's left hand moving back toward the operator's body and the sensed movement of the right hand controller corresponds to the operator's right hand moving forward away from the operator's body, and where, when in the gestural arm control mode, the surgical robotic system moves at least the portion of the robotic arm assembly to yaw an orientation of the chest plane to the left about the virtual chest pivot center with respect to the current field of view in response to the left yaw input.

In one embodiment, the plurality of gestural rotation inputs comprises or further comprises a pitch down input, in which the sensed movement of the hand controllers corresponds to the operator's hands tilting forward, and where, when in the gestural arm control mode, the surgical robotic system moves at least the portion of the robotic arm assembly to pitch the orientation of the chest plane downward about the virtual chest pivot center with respect to a current field of view of a current image displayed in response to the pitch down input; and a pitch up input in which the sensed movement of the hand controllers and the sensed movement of the operator's hands corresponds to the operator's hands tilting backward, and where, when in the gestural arm control mode, the surgical robotic system moves at least the portion of the robotic arm assembly to pitch the orientation of the chest plane upward about the virtual chest pivot center with respect to the current field of view in response to the pitch up input.

In one embodiment, the plurality of gestural rotation inputs comprises or further comprises a clockwise roll input, in which a sensed movement of a left hand controller corresponds to a left hand of the operator moving vertically up and a sensed movement of the right hand controller corresponds to a right hand of the operator moving vertically down, and where, when in the gestural arm control mode, the surgical robotic system moves at least the portion of the robotic arm assembly to rotate the robotic arm assembly clockwise about an axis parallel to the current viewing direction that passes through the virtual chest pivot center with respect to a current field of view of a current image displayed in response to the clockwise roll input; and a counter-clockwise roll input, in which the sensed movement of the left hand controller corresponds to the operator's left hand moving vertically down and the sensed movement of the right hand controller corresponds to the operator's right hand moving vertically up, and where, when in the gestural arm control mode, the surgical robotic system moves at least the portion of the robotic arm assembly to rotate the robotic arm assembly counter-clockwise about an axis parallel to the current viewing direction that passes through the virtual chest pivot center with respect to the current field of view in response to the counter-clockwise roll input.

In one embodiment, the first control mode is a physical activity arm control mode, in which one or more of: a magnitude of a translation of at least a portion of the robot arm assembly, a direction of the translation of at least the portion of the robotic arm assembly, a magnitude of a rotation of at least the portion of the robot arm assembly, and an axis of the rotation of at least the portion of the robotic arm assembly, depend, at least in part, on one or more of: a magnitude of the sensed movement of the hand controllers; a magnitude of a sensed change in separation between the hand controllers; a magnitude of a sensed change in lateral separation between the hand controllers; a direction of a movement of the hand controllers, and a sensed change in orientation of a line connecting the hand controllers in the first control input. The first control input corresponds to one of a plurality of different types of physical activity input.

In one embodiment, the plurality of different types of physical activity inputs includes a zoom input, in which the sensed movement hand controllers corresponds to a change in lateral separation between the hand controllers. Where the lateral separation between the hand controllers increases, the surgical robotic system moves at least the portion of the robotic arm assembly to move the location of the virtual chest pivot center forward in the current viewing direction with a magnitude of a displacement of the virtual chest pivot depending on a magnitude of the change in lateral separation in response to the first control input. Where the lateral separation between the hand controllers decreases, the surgical robotic system moves at least the portion of the robotic arm assembly to move the location of the virtual chest pivot center backward with respect to the current viewing direction with the magnitude of a displacement of the virtual chest pivot depending on the magnitude of the change in lateral separation in response to the first control input.

In one embodiment, the plurality of different types of physical activity inputs includes or further includes a wheel input, in which the sensed movement of the hand controllers correspond to an angular change in an orientation of a line connecting the hand controllers in a vertical plane. Where the change in orientation corresponds to clockwise rotation, the surgical robotic system moves at least the portion of the robotic arm assembly to rotate the orientation of the virtual chest to the right with respect to a current field of view of a current image displayed with a magnitude of the angular rotation of the virtual chest depending a magnitude of the angular change in the orientation of the line in response to the first control input. Where the change in orientation corresponds to a counter-clockwise rotation, the surgical robotic system moves at least the portion of the robotic arm assembly to rotate the orientation of the virtual chest to the left with respect to the current field of view with the magnitude of the angular rotation of the virtual chest depending on the magnitude of the angular change in the orientation of the line in response to the first control input.

In one embodiment, the first control mode is a gestural camera control mode. The first control input corresponds to one of a plurality of gestural rotation inputs.

In one embodiment, the plurality of gestural rotation inputs comprises or further comprises a right yaw input and a left yaw input. A sensed movement of a left hand controller in the right yaw input corresponds to a left hand of the operator moving forward away from the operator's body and a sensed movement of a right hand controller corresponds to a right hand of the operator moving back toward the operator's body, and where, when in the gestural camera control mode, the surgical robotic system moves at least a portion of the camera assembly to yaw an orientation of a direction of view of one or more cameras of the camera assembly to the right about a yaw rotation axis of the camera assembly with respect to a current field of view of a current image displayed in response to the right yaw input. The sensed movement of the operator's left hand in the left yaw input corresponds to the operator's left hand moving back toward the operator's body and the sensed movement of the operator's right hand corresponds to the operator's right hand forward away from the operator's body, and where, when in the gestural camera control mode, the surgical robotic system moves at least a portion of the camera assembly to yaw an orientation of a direction of view of the one or more cameras to the left about a yaw rotation axis of the camera assembly with respect to the current field of view of the current image displayed in response to the left yaw input.

In one embodiment, the plurality of gestural rotation inputs comprises or further comprises a pitch down input and a pitch up input. The sensed movement of the hand controllers in the pitch down input corresponds to the operator's hands tilting forward, and where, when in the gestural camera control mode, the surgical robotic system moves at least the portion of the camera assembly to pitch an orientation of the direction of view of the one or more cameras of the camera assembly downward in response to the pitch down input. The sensed movement of the hand controllers in the pitch up input corresponds to the operator's hands tilting backward, and where, when in the gestural camera control mode, the surgical robotic system moves at least the portion of the cameras assembly to pitch an orientation of the direction of view of the one or more cameras upward about the pitch axis of the camera assembly in response to the pitch up input.

In one embodiment, the plurality of gestural rotation inputs comprises or further comprises a clockwise roll input and a counter-clockwise roll input. The sensed movement of the hand controllers in the clockwise roll input corresponds to a left hand of the operator moving vertically up and a right hand of the operator moving vertically down, and where, when in the gestural camera control mode, the surgical robotic system moves at least the portion of the camera assembly to roll one or more cameras clockwise about an axis parallel to the current viewing direction in response to the clockwise roll input. The sensed movement of the hand controllers in the counter-clockwise roll input corresponds to the operator's left hand moving vertically down and the operator's right hand moving vertically up, and where, when in the gestural camera control mode, the surgical robotic system moves at least the portion of the camera assembly to roll the one or more cameras counter-clockwise about an axis parallel to the current viewing direction in response to the counter-clockwise roll input.

In one embodiment, the first control mode selection input is received via an input mechanism on one or both of the hand controllers.

In one embodiment, the first control mode selection input is received via a control on an operator console.

In one embodiment, the first control mode selection input is received via a foot pedal.

In one embodiment, the method further comprises receiving a second mode selection input and changing a current control mode of the surgical robotic system to a second control mode.

In one embodiment, the first control mode is a travel arm control mode and the second control mode is a camera control mode.

In one embodiment, the first control mode is a travel arm control mode and the second control mode is a different travel arm control mode.

In one embodiment, the first control mode is a camera control mode and the second control mode is an arm control mode.

In one embodiment, when in the second control mode, the surgical robotic system maintains the robotic assembly in a stationary position and a static configuration regardless of the hand controller movement.

In one embodiment, the second control mode is a default control mode.

In one embodiment, the second mode selection input corresponds to the operator releasing at least one operator control that was actuated and held or depressed by the operator to generate the first control input.

In one embodiment, the second mode selection input corresponds to the operator actuating a same operator control that was actuated by the operator to generate the first control input.

In one embodiment, the first mode selection input corresponds to the operator actuating a first operator control and the second mode selection input corresponds to the operator actuating a different second operator control.

In one embodiment, the method further comprises receiving a third mode selection input, and in response, changing a current control mode to a third control mode.

In one embodiment, the third control mode is the same as the second control mode.

In one embodiment, the third control mode is different from the first control mode and from the second control mode.

In one embodiment, the robotic surgical system further comprises a touchscreen display. The third control mode is a model manipulation control mode. The method further comprises displaying a representation of the robotic assembly in response to receipt of the third mode selection input; detecting a first touchscreen operator input selecting at least a portion of the displayed robotic assembly; detecting a second touchscreen operator input corresponding to the operator dragging the representation of the selected at least the portion of the robotic assembly to change a position and/or an orientation of the selected at least the portion of the robotic assembly in the representation displayed on the touchscreen; and in response to the detected second touchscreen operator input, moving one or more components of the robotic assembly corresponding to the selected at least one component while maintaining a stationary position of the instrument tips of the end effectors.

In a second aspect, the present disclosure provides a surgical robotic system for performing a surgery within an internal cavity of a subject. The surgical robotic system comprise hand controllers operated to manipulate the surgical robotic system, a computing unit configured to receive operator generated movement data from the hand controllers and to generate control signals in response based on a current control mode of the surgical robotic system, and receive a control mode selection input to change a current control mode of the surgical robotic system to a selected one of a plurality of control modes of the surgical robotic system in response, a camera assembly, a robotic arm assembly configured to be inserted into the internal cavity during use, the robotic assembly including a first robotic arm including a first end effector disposed at a distal end of the first robotic arm, and a second robotic arm including a second end effector disposed at a distal end of the second robotic arm, and an image display for outputting an image from the camera assembly.

In one embodiment, the first robotic arm and the second robotic arm define a virtual chest of robotic assembly, the virtual chest defined by a chest plane extending between a first pivot point of a most proximal joint of the first robotic arm, a second pivot point of a most proximal joint of the second robotic arm and a camera imaging center point of the camera assembly. The pivot center of the virtual chest lies midway along a line segment in the chest plane connecting the first pivot point of the first robotic arm and the second pivot point of the second robotic arm. The computing unit includes one or more processors configured to execute computer readable instructions to provide the plurality of control modes of the surgical robotic system. The plurality of control modes includes a travel arm control mode and/or a camera control mode. Where the surgical robotic system is in a camera control mode and a first control input is received from hand controllers regarding a sensed movement of the operator's hands, in response to the first control input, the surgical robotic system moves at least a portion of the camera assembly to change an orientation and/or a positon of at least one camera of the camera assembly with respect to a current viewing direction while keeping the robotic arm assembly stationary. Where the surgical robotic system is in a travel arm control mode, and the first control input is received from hand controllers regarding the sensed movement of the operator's hands, in response to receiving the first control input, the surgical robotic system moves at least a portion of the robotic arm assembly to change a location of the virtual chest pivot center and/or an orientation the virtual chest with respect to the current viewing direction while maintaining stationary instrument tips of the end effectors disposed at distal ends of the robotic arms.

In a third aspect, the present disclosure provides a non-transitory computer readable medium having instructions stored thereon for controlling a robotic assembly of a surgical robotic system. When the instructions are executed by a processor, the instructions cause the processor to control the surgical robotic system to carry out methods and embodiments described herein.

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. These and other features and advantages of the present invention will be more fully understood by reference to the following detailed description in conjunction with the attached drawings in which like reference numerals refer to like elements throughout the different views.

FIG. 1 schematically depicts a surgical robotic system in accordance with some embodiments.

FIG. 2A is a perspective view of a patient cart including a robotic support system coupled to a robotic subsystem of the surgical robotic system in accordance with some embodiments.

FIG. 2B is a perspective view of an example operator console of a surgical robotic system of the present disclosure in accordance with some embodiments.

FIG. 3A schematically depicts a side view of a surgical robotic system performing a surgery within an internal cavity of a subject in accordance with some embodiments.

FIG. 3B schematically depicts a top view of the surgical robotic system performing the surgery within the internal cavity of the subject of FIG. 3A in accordance with some embodiments.

FIG. 4A is perspective view of a single robotic arm subsystem in accordance with some embodiments.

FIG. 4B is perspective side view of a single robotic arm of the single robotic arm subsystem of FIG. 4A in accordance with some embodiments.

FIG. 5 is a perspective front view of a camera assembly and a robotic arm assembly in accordance with some embodiments.

FIG. 6 is a flowchart illustrating steps for controlling a robotic assembly carried out by a surgical robotic system in accordance with some embodiments.

FIG. 7A schematically depicts hand gestures for a pullback input and a push forward input in a gestural arm control mode in accordance with some embodiments.

FIG. 7B schematically depicts a top view of movements of a robotic arm assembly in response to the pullback input and the push forward input of FIG. 7A in accordance with some embodiments.

FIG. 8A schematically depicts hand gestures for a horizontal input in a gestural arm control mode in accordance with some embodiments.

FIG. 8B schematically depicts a top view of a robotic arm assembly in response to the horizontal input of FIG. 8A in accordance with some embodiments;

FIG. 9A schematically depicts hand gestures for a vertical input in a gestural arm control mode in accordance with some embodiments.

FIG. 9B schematically depicts the movements of a robotic arm assembly in response to the vertical input of FIG. 9B in accordance with some embodiments.

FIG. 10A schematically depicts hand gestures for a right yaw input and a left yaw input in a gestural arm control mode in accordance with some embodiments.

FIG. 10B schematically depicts movements of robotic arm assembly in response to the right yaw input and left yaw input of FIG. 10A in accordance with some embodiments.

FIG. 11A schematically depicts hand gestures for a pitch down input and a pitch up input in a gestural arm control mode in accordance with some embodiments;

FIG. 11B schematically depicts movements of the robotic arm assembly in response to the pitch down input and the pitch up input of FIG. 11A in accordance with some embodiments.

FIG. 12A schematically depicts hand gestures for a clockwise roll input and a counter-clockwise roll input in a gestural arm control mode in accordance with some embodiments;

FIG. 12B schematically depicts the movements of the robotic arm assembly in response to the clockwise roll input and the counter-clockwise roll input in accordance with some embodiments;

FIG. 13A schematically depicts hand gestures for a right yaw input and a left yaw input in a gestural camera control mode in accordance with some embodiments.

FIG. 13B schematically depicts movements of a camera assembly in response to the right yaw input and the left yaw input in FIG. 13A in accordance with some embodiments.

FIG. 13C schematically depicts hand gestures for a pitch down input and a pitch up input in a gestural camera control mode in accordance with some embodiments.

FIG. 13D schematically depicts movements of a camera assembly in response to the pitch down input and the pitch up input in FIG. 13C in accordance with some embodiments.

FIG. 13E schematically depicts hand gestures for a clockwise roll input and a counter-clockwise roll input in a gestural camera control mode in accordance with some embodiments.

FIG. 13F schematically depicts movements of a camera assembly in response to the clockwise roll input and the counter-clockwise roll input in FIG. 13E in accordance with some embodiments.

FIGS. 14A-14D schematically depict hand gestures for example zoom inputs in a physical activity control mode and movements of a robotic arm assembly in response to the zoom inputs in accordance with some embodiments.

FIGS. 15A-15D schematically depict hand gestures for wheel inputs corresponding to a clockwise rotation in a physical activity mode and movements of a robotic arm assembly in response to the wheel inputs in accordance with some embodiments.

FIG. 16A depicts a top view of a robotic assembly in an abdominal cavity of a subject with the robotic assembly extending in an inferior direction with respect to the subject in accordance with some embodiments.

FIG. 16B depicts a top view of the robotic assembly of FIG. 16A in the abdominal cavity with the robotic assembly changing an orientation of a virtual chest to the right with respect to a field of view of a current image displayed in accordance with some embodiments.

FIG. 16C depicts a top view of the robotic assembly of FIG. 16B in the abdominal cavity with the robotic assembly further changing the orientation of the virtual chest to the right with respect to the direction of FIG. 16B in accordance with some embodiments.

FIG. 17A depicts a top view of the robotic assembly of FIG. 16A in the abdominal cavity with the robotic assembly extending in a more lateral direction with respect to the subject in accordance with some embodiments.

FIG. 17B depicts a top view of the robotic assembly of FIG. 17A in the abdominal cavity with the robotic assembly repositioning a camera assembly more close to end effectors to in accordance with some embodiments.

FIG. 17C depicts a top view of the robotic assembly of FIG. 17B in the abdominal cavity with the robotic assembly repositioning end effectors in an anterior direction with respect to the subject in accordance with some embodiments.

FIG. 17D depicts a top view of the robotic assembly of FIG. 18C in the abdominal cavity with the robotic assembly repositioning camera assembly more close to the end effectors to in accordance with some embodiments.

FIG. 18A depicts a top view of a robotic assembly having a camera assembly forward facing in accordance with some embodiments.

FIG. 18B depicts a top view of a robotic assembly having a camera assembly left facing in accordance with some embodiments.

FIG. 18C depicts a top view of a robotic assembly having a camera assembly right facing in accordance with some embodiments.

FIG. 19A depicts a top view of a robotic assembly having a camera assembly backward facing in accordance with some embodiments.

FIG. 19B depicts a top view of the robotic assembly of FIG. 19A with the robotic assembly changing an orientation of a virtual chest in accordance with some embodiments.

FIG. 20 is a flowchart for performing a model manipulation control mode carried out by a surgical robotic system of the present disclosure in accordance with some embodiments.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It may be understood that various alternatives to the embodiments of the invention described herein may be employed.

As used in the specification and claims, the singular form “a,” “an,” and “the” include plural references unless the context clearly dictates otherwise.

Some embodiments disclosed herein are implemented on, employ, or are incorporated into a surgical robotic system that includes a camera assembly having at least three articulating degrees of freedom and two or more robotic arms each having at least six articulating degrees of freedom and an additional degree of freedom corresponding to the movement of an associated end-effector (e.g., grasper, manipulator, and the like). In some embodiments, the camera assembly when mounted within a subject (e.g., a patient) can be moved or rotated in a pitch or yaw direction about 180 degrees such that the camera assembly can view rearwardly back towards the insertion site. As such, the camera assembly and the robotic arms can view and operate dexterously forward (e.g., away from the insertion site), to each side, in an upward or downward direction, as well as in the rearward direction to view backwards towards the insertion site. The robot arms and the camera assembly can also move in the roll, pitch and yaw directions.

Control modes described herein are particularly advantageous in a surgical robotic system having greater maneuverability than a conventional system. For example, many conventional surgical robotic systems having two robotic arms and fewer degrees of freedom per arm may not be able to change a position or an orientation of a virtual chest of the robotic arms while keeping instrument tips of end effectors of the robotic arms stationary. As another example, cameras of many conventional surgical robotic systems may have only have degrees of freedom associated with movement of a support for the camera extending through a trocar and may have no independent degrees of freedom for movement relative to the support.

This large number of degrees of freedom in surgical robotic systems described herein, in comparison to some conventional surgical robotic systems, enables movements of a robotic arm assembly and orientations of a robotic arm assembly not possible with some conventional surgical robotic arms and enables movements of a camera of a robotic camera assembly not possible in cameras for some conventional robotic surgical systems.

Some surgical robotic systems described herein employ control in which movement of a left hand controller causes a corresponding scaled-down movement of a distal end of the left robotic arm and movement of a right hand controller causes a corresponding scaled-down movement of a distal end of the right robotic arm. This control is referred to as scaled-down arm control herein. Movement of the hand controllers using this type of control cannot change a position and/or an orientation of the chest of the robotic arms without also changing positions of instrument tips of the end effectors at distal ends of the robotic arms. Further, with this type of control, some types of change in orientation of the chest of the robotic arms. In this type of control, a direction of view or orientation of a camera may not controlled by movements of the arm controllers, but instead may be controlled by another operator input.

The present disclosure provides systems and methods for controlling a robotic assembly of a surgical robotic system when at least a portion of the robotic assembly is disposed in an interior cavity of a subject. The robotic assembly includes a camera assembly and a robotic arm assembly including a first robotic arm and a second robotic arm defining a virtual chest of the robotic arm assembly. As used herein, a distal end of a robotic arm extends away from virtual chest of a robotic arm assembly. Some methods and systems described herein provide or employ multiple different control modes of the surgical robotic system, each control mode using sensed movement of hand controllers to control the robotic arm assembly and/or the camera assembly, and changing from a current control mode to a different selected control mode based on operator input. In some methods and systems, the multiple different control modes, which may be described as a plurality of control modes, include at least one control mode in which a position and/or an orientation of: at least a portion of the camera assembly, of at least a portion of the robotic arm assembly, or both, are changed in response to movement of the hand controllers, while maintaining a stationary position of instrument tips of end effectors disposed at distal ends of the robotic arms. In some systems and methods, the multiple different control modes includes at least one travel arm control mode, at least one camera control mode, or both. In a travel arm control mode, the surgical robotic system moves at least a portion of the robotic arm assembly to change a location of a virtual chest pivot center and/or an orientation a virtual chest of the robotic arm assembly with respect to a current viewing direction or a current field of view in response to movement of the hand controllers while maintaining a stationary position of instrument tips of end effectors disposed at distal ends of the robotic arms. In a camera mode control mode, the surgical robotic system moves at least a portion of the camera assembly to change an orientation of a direction of view in response to movement of hand controllers maintaining a stationary position of instrument tips of the robotic arms.

In some methods and systems, the one or more travel arm control modes include one or both of a travel gestural arm control mode and a physical activity arm control mode. In a travel gestural arm control mode, movement of arm controllers corresponding to one of a plurality of gestural translation inputs causes the surgical robotic system to move at least a portion of the robotic arm assembly to change the location of the virtual chest pivot center while maintaining the stationary position of the instrument tips of the end effectors in response to the first control input, and movement of arm controllers corresponding to one of a plurality of gestural rotation inputs causes the surgical robotic system to move at least a portion of the robotic arm assembly to change the orientation of the virtual chest with respect to the current viewing direction while maintaining a stationary position of instrument tips of the end effectors. In some embodiments, the plurality of gestural translation inputs includes: a pullback input, a push forward input, a horizontal input, a vertical input, or any combination of the aforementioned. In some embodiments, the plurality of gestural translation inputs includes: a right yaw input, a left yaw input, a pitch down input, a pitch up input, a clockwise roll input, a counter-clockwise roll input, or any combination of the aforementioned.

In a physical activity arm control mode, movement of hand controllers corresponding to one of a plurality of different types of physical activity arm control inputs causes the surgical robotic system to move at least a portion of the robotic arm assembly to change a location of the virtual chest pivot center and/or an orientation the virtual chest of the robotic arm assembly with respect to a current viewing direction or a current field of view in response to movement of the hand controllers while maintaining a stationary position of the instrument tips of the end effectors. In the physical activity mode one or more of: a magnitude of a translation of at least a portion of the robot arm assembly, a direction of the translation of at least the portion of the robotic arm assembly, a magnitude of a rotation of at least the portion of the robot arm assembly, and an axis of the rotation of at least the portion of the robotic arm assembly, depend, at least in part, on one or more of: a magnitude of the sensed movement of the hand controllers; a magnitude of a sensed change in separation between the hand controllers; a magnitude of a sensed change in lateral separation between the hand controllers; a direction of a movement of the hand controllers, and a sensed change in orientation of a line connecting the hand controllers in the first control input. In some embodiments, the plurality of types of physical activity control inputs includes: a zoom input, a wheel input for yawing, a directional pull input, a direction push input, or any combination of the aforementioned.

In some embodiments, aspects of the physical activity arm control mode may be incorporated into the gestural travel arm control mode and one or more of: a magnitude of a translation of at least a portion of the robot arm assembly, a direction of the translation of at least the portion of the robotic arm assembly, a magnitude of a rotation of at least the portion of the robot arm assembly, and an axis of the rotation of at least the portion of the robotic arm assembly, depend, at least in part, on one or more of: a magnitude of the sensed movement of the hand controllers; a magnitude of a sensed change in separation between the hand controllers; a magnitude of a sensed change in lateral separation between the hand controllers; a direction of a movement of the hand controllers, and a sensed change in orientation of a line connecting the hand controllers in the first control input.

In a gestural camera control mode, movement of hand controllers corresponding to one of a plurality of gestural rotation inputs causes the surgical robotic system to change an orientation and/or of at least one camera of the camera assembly with respect to the current viewing direction while keeping the robotic arm assembly stationary. In some embodiments, the plurality of gestural rotation inputs includes one or more of: a pitch up input, a pitch down input, a yaw left input, a yaw right input, a clockwise roll input, a counter-clockwise roll input, or any combination of the aforementioned. In some embodiments, selected aspects of the physical activity arm control mode may be incorporated into the gestural camera control mode, and a magnitude of a rotation or change in orientation of the camera and/or an axis of rotation for a change in orientation of the camera, depend, at least in part, on one or more of: a magnitude of the sensed movement of the hand controllers; a direction or directions of movement of the hand controllers, and a sensed change in orientation of a line connecting the hand controllers.

Some methods and systems described herein provide or employ a control mode of a surgical robotic system, which is referred to herein as a model manipulation mode, in which a representation of the robotic assembly is displayed on a touchscreen. In the model manipulation mode, a detection of a touch selecting of at least a portion of the representation of the robotic assembly and dragging the selected portion of the representation of the robotic assembly causes a change a position and/or an orientation of the selected at least the portion of the robotic assembly in the representation displayed on the touchscreen; and moving one or more components of the robotic assembly corresponding to the selected at least one component while maintaining a stationary position of the instrument tips of the end effectors.

In some embodiments, systems and methods may incorporate any or all of the control modes disclosed herein and mechanisms for the operator to switch between control modes.

Providing a plurality of different control modes employing hand controllers enables an operator to use movements of hand controllers to perform different functions in different control modes. Some of these functions, like independent control of a camera assembly, would require an operator to use other operator controls that may or may not be associated with a hand controller, like separate switches, a separate joystick, or separate buttons, to accomplish these other functions. Switching from motion of hand controllers to other operator controls and back for accomplishing various functions can slow down a procedure, and may require the operator removing his or her hand from a hand controller to access the other cooperator controls. Further, switching from motion of hand controllers to other operator controls may interrupt a flow of work during a surgical procedure, and may increase complexity of use of a system. Thus, enabling additional functionality associated with movement of hand controls via switching control modes may provide a more streamlined operator experience and increased operator efficiency.

Maintaining instrument tip positions while changing an orientation of a virtual chest plane and/or a position of a chest pivot center of an arm assembly may ensure that instrument tips will not inadvertently move causing damage to a patient while reconfiguring or reorienting the arm assembly in some embodiments. In some embodiments, a user may switch between a travel arm control mode and control mode employing scaled-down arm control, which may be referred to herein as a scaled-down arm control mode. By switching between the travel arm control mode and the scaled-down arm control mode, an operator may extend the robotic arms to “reach” and position the end effectors as desired, and then switch to a travel arm control mode to “pull” to reposition and/or reorient the base relative to the end effectors. The operator may switch into the camera mode to obtain a view in different directions, and/or reenter the scaled-down arm control mode to reposition the end effectors in a new location. Through this switching between modes the operator can traverse an internal cavity and control an orientation and configuration of the arm assembly.

Travel control modes enable reorientation and reconfiguration of the arm assembly within an interior cavity, while reducing or eliminating a risk that that motion of instrument tips of the end effectors during the reorientation and reconfiguration would damage the body cavity.

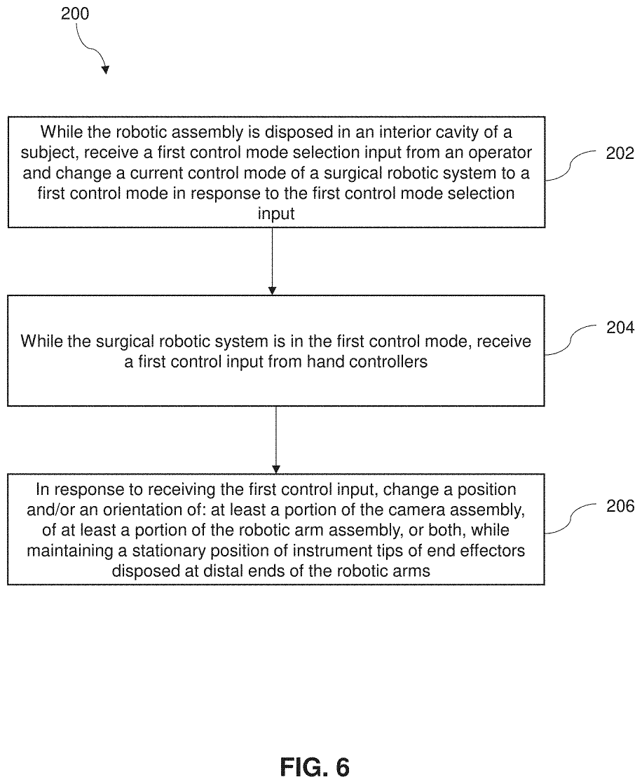

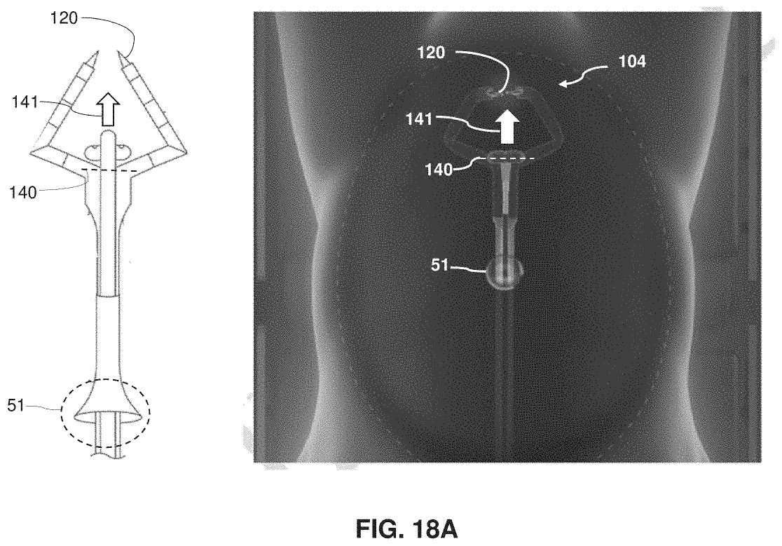

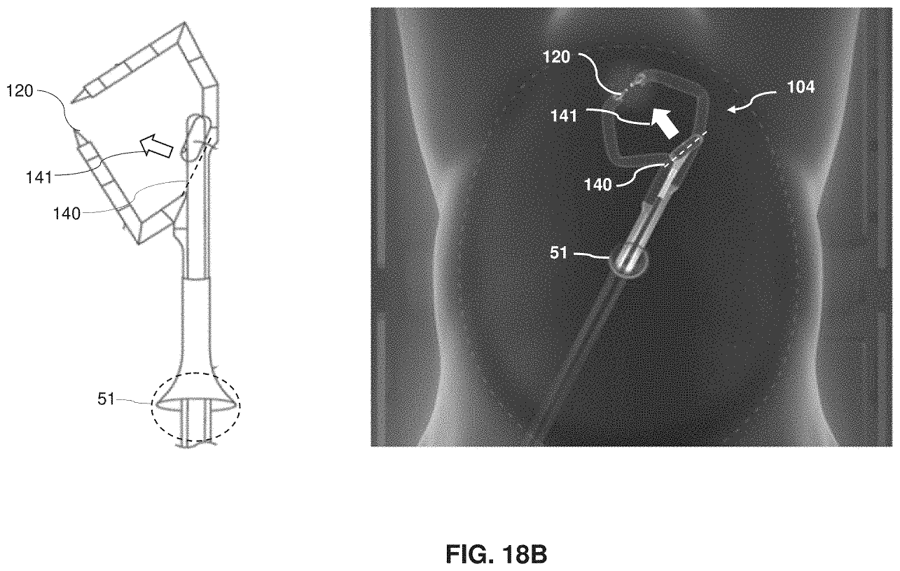

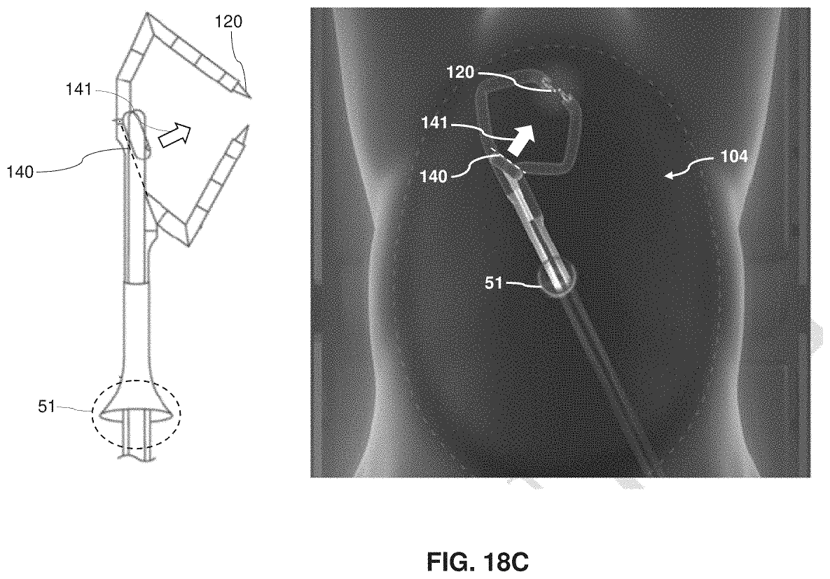

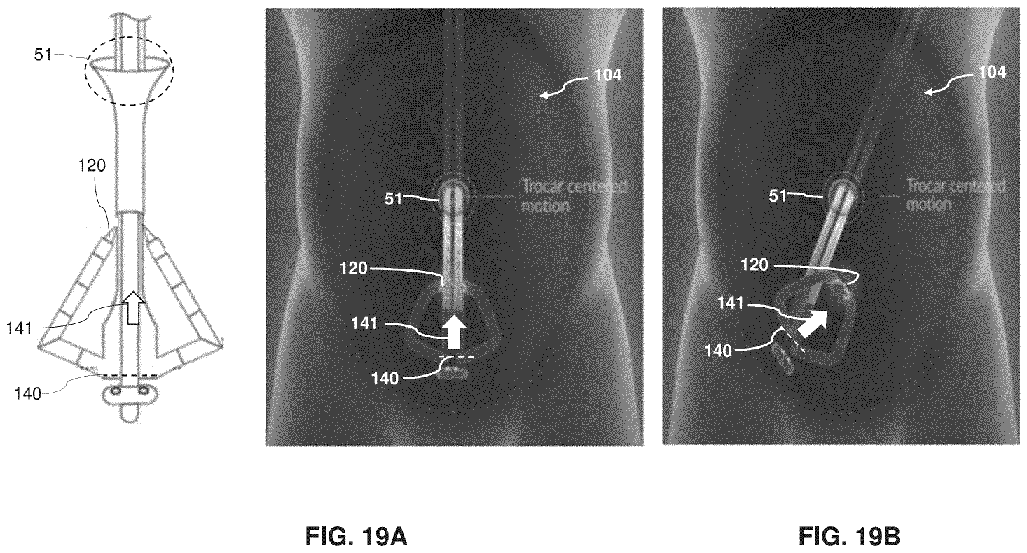

Prior to addressing control modes in detail with respect to FIGS. 6-20, a description is provided of example surgical robotics systems and robotic assemblies for implementing embodiments described herein

### Surgical Robotic Systems

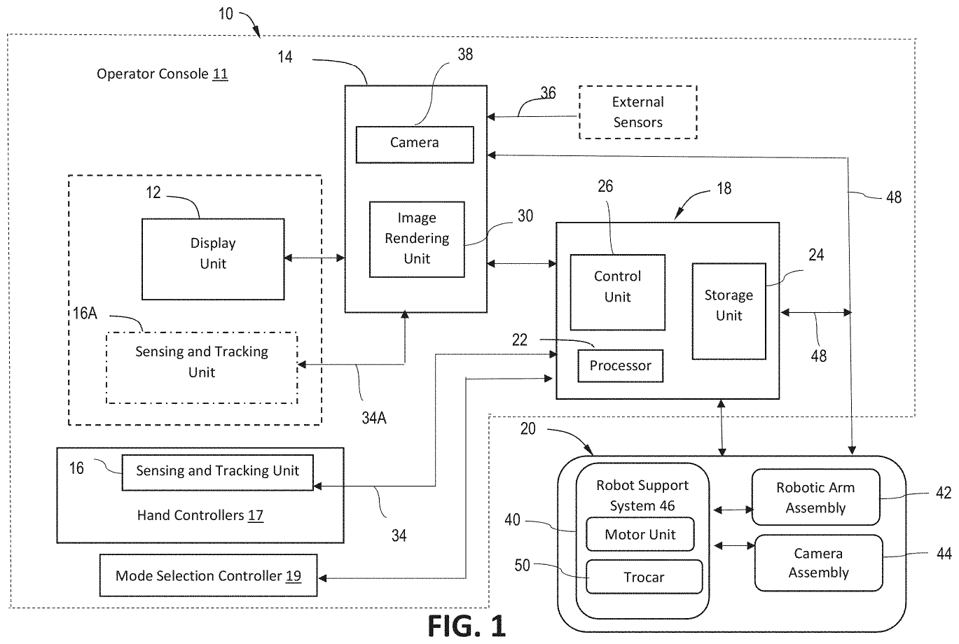

Turning to the drawings, FIG. 1 is a schematic illustration of a surgical robotic system 10 in accordance with some embodiments of the present disclosure. The surgical robotic system 10 includes an operator console 11 and a robotic assembly 20.

The operator console 11 includes a display device or unit 12, an image computing unit 14, which may be a virtual reality (VR) computing unit, hand controllers 17 having a sensing and tracking unit 16, a computing unit 18, and a mode selection controller 19.

The display unit 12 can be any selected type of display for displaying information, images or video generated by the image computing unit 14, the computing unit 18, and/or the robotic assembly 20. The display unit 12 can include or form part of, for example, a head-mounted display (HMD), an augmented reality (AR) display (e.g., an AR display, or AR glasses in combination with a screen or display), a screen or a display, a two-dimensional (2D) screen or display, a three-dimensional (3D) screen or display, and the like. The display unit 12 can also include an optional sensing and tracking unit 16A. In some embodiments, the display unit 12 can include an image display for outputting an image from a camera assembly 44 of the robotic assembly 20.

In some embodiments, if the display unit 12 includes an HMD device, an AR device that senses head position, or another device that employs an associated sensing and tracking unit 16A, the HMD device or head tracking device generates tracking and position data 34A that is received and processed by image computing unit 14. In some embodiments, the HMD, AR device, or other head tracking device can provide an operator (e.g., a surgeon, a nurse or other suitable medical professional) with a display that is at least in part coupled or mounted to the head of the operator, lenses to allow a focused view of the display, and the sensing and tracking unit 16A to provide position and orientation tracking of the operator's head. The sensing and tracking unit 16A can include for example accelerometers, gyroscopes, magnetometers, motion processors, infrared tracking, eye tracking, computer vision, emission and sensing of alternating magnetic fields, and any other method of tracking at least one of position and orientation, or any combination thereof. In some embodiments, the HMD or AR device can provide image data from the camera assembly 44 to the right and left eyes of the operator. In some embodiments, in order to maintain a virtual reality experience for the operator, the sensing and tracking unit 16A, can track the position and orientation of the operator's head, generate tracking and position data 34A, and then relay the tracking and position data 34A to the image computing unit 14 and/or the computing unit 18 either directly or via the image computing unit 14.

The hand controllers 17 are configured to sense a movement of the operator's hands and/or arms to manipulate the surgical robotic system 10. The hand controllers 17 can include the sensing and tracking unit 16, circuity, and/or other hardware. The sensing and tracking unit 16 can include one or more sensors or detectors that sense movements of the operator's hands. In some embodiments, the one or more sensors or detectors that sense movements of the operator's hands are disposed in a pair of hand controllers that are grasped by or engaged by hands of the operator. In some embodiments, the one or more sensors or detectors that sense movements of the operator's hands are coupled to the hands and/or arms of the operator. For example, the sensors of the sensing and tracking unit 16 can be coupled to a region of the hand and/or the arm, such as the fingers, the wrist region, the elbow region, and/or the shoulder region. If the HMD is not used, then additional sensors can also be coupled to a head and/or neck region of the operator in some embodiments. If the operator employs the HMD, then the eyes, head and/or neck sensors and associated tracking technology can be built-in or employed within the HMD device, and hence form part of the optional sensor and tracking unit 16A as described above. In some embodiments, the sensing and tracking unit 16 can be external and coupled to the hand controllers 17 via electricity components and/or mounting hardware.

In some embodiments, the sensing and tracking unit 16 can employ sensors coupled to the torso of the operator or any other body part. In some embodiments, the sensing and tracking unit 16 can employ in addition to the sensors an Inertial Momentum Unit (IMU) having for example an accelerometer, gyroscope, magnetometer, and a motion processor. The addition of a magnetometer allows for reduction in sensor drift about a vertical axis. In some embodiments, the sensing and tracking unit 16 also include sensors placed in surgical material such as gloves, surgical scrubs, or a surgical gown. The sensors can be reusable or disposable. In some embodiments, sensors can be disposed external of the operator, such as at fixed locations in a room, such as an operating room. The external sensors can generate external data 36 that can be processed by the computing unit 18 and hence employed by the surgical robotic system 10.

The sensors generate position and/or orientation data indicative of the position and/or orientation of the operator's hands and/or arms. The sensing and tracking units 16 and/or 16A can be utilized to control movement (e.g., changing a position and/or an orientation) of the camera assembly 44 and a robotic arm assembly 42 of the robotic assembly 20. The tracking and position data 34 generated by the sensing and tracking unit 16 can be conveyed to the computing unit 18 for processing by a processor 22.

The computing unit 18 can determine or calculate, from the tracking and position data 34 and 34A, the position and/or orientation of the operator's hands or arms, and in some embodiments of the operator's head as well, and convey the tracking and position data 34 and 34A to the robotic assembly 20. The tracking and position data 34, 34A can be processed by the processor 22 and can be stored for example in the storage unit 24. The tracking and position data 34A can also be used by the control unit 26, which in response can generate control signals for controlling movement of the robotic arm assembly 42 and/or the camera assembly 44. For example, the control unit 26 can change a position and/or an orientation of at least a portion of the camera assembly 44, of at least a portion of the robotic arm assembly 42, or both. In some embodiments, the control unit 26 can also adjust the pan and tilt of the camera assembly 44 to follow the movement of the operator's head.

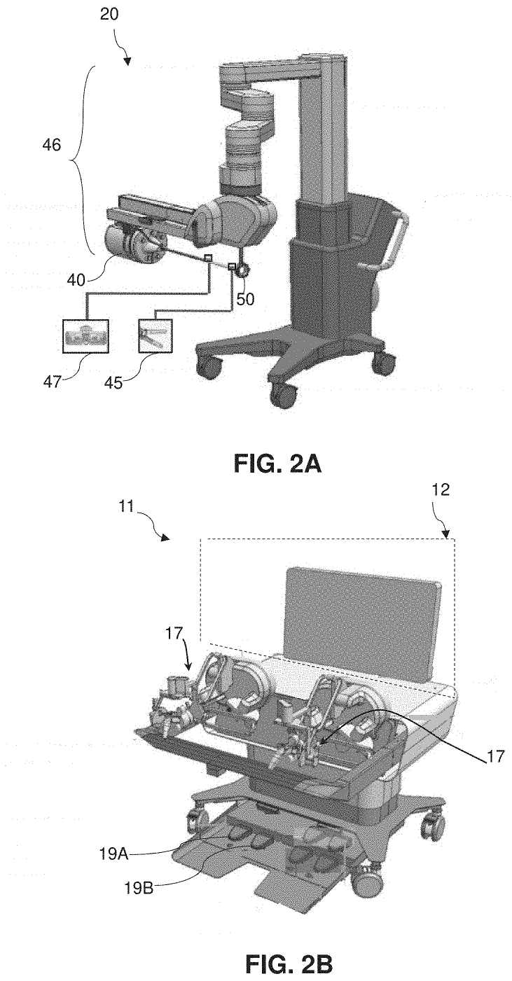

The mode selection controller 19 is used to select a control model from multiple control modes. Examples of control modes can include a travel arm control mode, a camera control mode, a physical activity control model, a model manipulation control mode, and a default mode. In some embodiments, the mode selection controller 19 can communicate with the hand controllers 17 to determine a control mode selection input. In some embodiments, the model selection controller 19 can obtain input from one or more foot pedals. The operator can depress and hold a specific foot pedal to enter a specific control mode, or tap a specific foot pedal to enter and/or exit a specific control mode. In some embodiments, the model selection controller 19 can also or alternatively obtain input from one or buttons, toggles, and/or switches that may be included in or on the hand controllers 17. The computing unit 18 can receive a first control mode selection input from the mode selection controller 19 and change a current control mode of the surgical robotic system 10 to a first control mode (e.g., a travel arm control mode, a camera control mode, a physical activity mode, model manipulation control mode, a default mode, or the like) in response to the first control mode selection input. The computing unit 18 can receive a first control input from the hand controllers 17. The computing unit 18 can change a position and/or an orientation of: at least a portion of the camera assembly 44, of at least a portion of the robotic arm assembly 42, or both, while maintaining a stationary position of instrument tips of end effectors disposed at distal ends of the robotic arms of the robotic arm assembly 42. Examples are further described with respect to FIGS. 2B, and 6-13.

The robotic assembly 20 can include a robot support system (RSS) 46 having a motor unit 40 and a trocar 50, the robotic arm assembly 42 and the camera assembly 44. The robotic arm assembly 42 and the camera assembly 44 can form part of a single support axis robot system, such as that disclosed and described in U.S. Pat. No. 10,285,765, or can form part of a split arm (SA) architecture robot system, such as that disclosed and described in PCT Patent Application No. PCT/US2020/039203.

The robotic assembly 20 can employ multiple different robotic arms that are deployable along different or separate axes. In some embodiments, the camera assembly 44, which can employ multiple different camera elements, can also be deployed along a common separate axis. Thus, the surgical robotic system 10 can employ multiple different components, such as a pair of separate robotic arms and the camera assembly 44, which are deployable along different axes. In some embodiments, the robotic arm assembly 42 and the camera assembly 44 are separately manipulatable, maneuverable, and movable. The robotic assembly 20, which includes the robotic arm assembly 42 and the camera assembly 44, is disposable along separate manipulatable axes, and is referred to herein as an SA architecture. The SA architecture is designed to simplify and increase efficiency of the insertion of robotic surgical instruments through a single trocar at a single insertion point or site, while concomitantly assisting with deployment of the surgical instruments into a surgical ready state, as well as the subsequent removal of the surgical instruments through a trocar 50 as further described below.

The RSS 46 can include the motor unit 40 and the trocar 50. The RSS 46 can further include a support member that supports the motor unit 40 coupled to a distal end thereof. The motor unit 40 in turn can be coupled to the camera assembly 44 and to each of the robotic arm assembly 42. The support member can be configured and controlled to move linearly, or in any other selected direction or orientation, one or more components of the robotic assembly 20. In some embodiments, the RSS 46 can be free standing. In some embodiments, the RSS 46 can include the motor unit 40 that is coupled to the robotic assembly 20 at one end and to an adjustable support member or element at an opposed end.

The motor unit 40 can receive the control signals generated by the control unit 26. The motor unit 40 can include gears, one or more motors, drivetrains, electronics, and the like, for powering and driving the robot arm assembly 42 and the cameras assembly 44 separately or together. The motor unit 40 can also provide mechanical power, electrical power, mechanical communication, and electrical communication to the robotic arm assembly 42, the camera assembly 44, and/or other components of the RSS 46 and robotic assembly 20. The motor unit 40 can be controlled by the computing unit 18. The motor unit 40 can thus generate signals for controlling one or more motors that in turn can control and drive the robotic arm assembly 42, including for example the position and orientation of each articulating joint of each robotic arm, as well as the camera assembly 44. The motor unit 40 can further provide for a translational or linear degree of freedom that is first utilized to insert and remove each component of the robotic assembly 20 through the trocar 50. The motor unit 40 can also be employed to adjust the inserted depth of each robotic arm assembly 42 when inserted into the patient 100 through the trocar 50.

The trocar 50 is a medical device that can be made up of an awl (which may be a metal or plastic sharpened or non-bladed tip), a cannula (essentially a hollow tube), and a seal. The trocar can be used to place at least a portion of the robotic assembly 20 in an interior cavity of a subject (e.g., a patient) and can withdraw gas and/or fluid from a body cavity. The robotic assembly 20 can be inserted through the trocar to access and perform an operation in vivo in a body cavity of a patient. The robotic assembly 20 can be supported by the trocar with multiple degrees of freedom such that the robotic arm assembly 42 and the camera assembly 44 can be maneuvered within the patient into a single position or multiple different positions.

In some embodiments, the RSS 46 can further include an optional controller for processing input data from one or more of the system components (e.g., the display 12, the sensing and tracking unit 16, the robot arm assembly 42, the camera assembly 44, and the like), and for generating control signals in response thereto. The motor unit 40 can also include a storage element for storing data.

The robot arm assembly 42 can be controlled to follow the scaled-down movement or motion of the operator's arms and/or hands as sensed by the associated sensors, which is referred to herein as a scaled-down arm control mode. The robot arm assembly 42 includes a first robotic arm including a first end effector having an instrument tip disposed at a distal end of the first robotic arm, and a second robotic arm including a second end effector having an instrument tip disposed at a distal end of the second robotic arm. In some embodiments, the robot arm assembly 42 can have portions or regions that can be associated with movements associated with the shoulder, elbow, and wrist joints as well as the fingers of the operator. For example, the robotic elbow joint can follow the position and orientation of the human elbow, and the robotic wrist joint can follow the position and orientation of the human wrist. The robot arm assembly 42 can also have associated therewith end regions that can terminate in end-effectors that follow the movement of one or more fingers of the operator in some embodiments, such as for example the index finger as the user pinches together the index finger and thumb. In some embodiments, while the robotic arms of the robot arm assembly 42 follow movement of the arms of the operator in some control modes (e.g., in a scaled-down arm control mode), the robotic shoulders are fixed in position in such control modes. In some embodiments, the position and orientation of the torso of the operator are subtracted from the position and orientation of the operator's arms and/or hands. This subtraction allows the operator to move his or her torso without the robot arms moving.

The camera assembly 44 is configured to provide the operator with image data 48, such as for example a live video feed of an operation or surgical site, as well as enable the operator to actuate and control the cameras forming part of the camera assembly 44. In some embodiments, the camera assembly 44 can include one or more cameras (e.g., a pair of cameras), the optical axes of which are axially spaced apart by a selected distance, known as the inter-camera distance, to provide a stereoscopic view or image of the surgical site. In some embodiments, the operator can control the movement of the cameras via movement of the hands via sensors coupled to the hands of the operator or via hand controllers grasped or held by hands of the operator, thus enabling the operator to obtain a desired view of an operation site in an intuitive and natural manner. In some embodiments, the operator can additionally control the movement of the camera via movement of the operator's head. The camera assembly 44 is movable in multiple directions, including for example in yaw, pitch and roll directions relative to a direction of view. In some embodiments, the components of the stereoscopic cameras can be configured to provide a user experience that feels natural and comfortable. In some embodiments, the interaxial distance between the cameras can be modified to adjust the depth of the operation site perceived by the operator.

The image or video data 48 generated by the camera assembly 44 can be displayed on the display unit 12. In embodiments in which the display unit 12 includes a HMD, the display can include the built-in sensing and tracking unit 16A that obtains raw orientation data for the yaw, pitch and roll directions of the HMD as well as positional data in Cartesian space (x, y, z) of the HMD. In some embodiments, positional and orientation data regarding an operator's head may be provided via a separate head-tracking unit. In some embodiments, the sensing and tracking unit 16A may be used to provide supplementary position and orientation tracking data of the display in lieu of or in addition to the built-in tracking system of the HMD. In some embodiments, no head tracking of the operator is used or employed.

The image data 48 generated by the camera assembly 44 can be conveyed to the imaging computing unit 14, which may be a VR computing unit, and can be processed by the image computing unit or image rendering unit 30, which may be a VR image rendering unit in some embodiments. The image data 48 can include still photographs or image data as well as video data in some embodiments. The image-rendering unit 30 can include suitable hardware and software for processing the image data and then rendering the image data for display by the display unit 12. Further, the rendering unit 30 can combine the image data received from the camera assembly 44 with information associated with the position and orientation of the cameras in the camera assembly, as well as information associated with the position and orientation of the head of the operator in embodiments that track the operator's head. With this information, the image-rendering unit 30 can generate an output video or image-rendering signal and transmit this signal to the display unit 12. That is, the image-rendering unit 30 renders the position and orientation readings of the hand controllers 17, and the head position of the operator for embodiments that track operator head position, for display in the display unit 12.

In some embodiments in which the image computing unit 14 is a VR computing unit, the image computing unit 14 can also include a VR camera unit 38 that can generate one or more virtual cameras in a virtual world, and which can be employed by the surgical robotic system 10 to render the images for the HMD. This ensures that the VR camera unit 38 always renders the same views that the operator wearing the HMD sees to a cube map. In some embodiments, a single VR camera can be used, and, in another embodiment, separate left and right eye VR cameras can be employed to render onto separate left and right eye cube maps in the display to provide a stereo view. The field of view (FOV) setting of the VR camera can self-configure itself to the FOV published by the camera assembly 44. In addition to providing a contextual background for the live camera views or image data, the cube map can be used to generate dynamic reflections on virtual objects. This effect allows reflective surfaces on virtual objects to pick up reflections from the cube map, making these objects appear to the user as if they're actually reflecting the real-world environment.

FIG. 2A depicts an example robotic assembly 20 of a surgical robotic system 10 of the present disclosure incorporated into or mounted onto a mobile patient cart in accordance with some embodiments. In some embodiments, the robotic assembly 20 includes the RSS 46, which, in turn includes the motor unit 40, the robotic arm assembly 42 having end-effectors 45, the camera assembly 44 having one or more cameras 47, and may also include the trocar 50.

FIG. 2B depicts an example of an operator console 11 of the surgical robotic system 10 of the present disclosure in accordance with some embodiments. The operator console 11 includes a display unit 12, hand controllers 17, and mode selection controllers 19, to select a control mode. In some embodiments, at least some mode selection controllers are incorporated to the hand controllers.

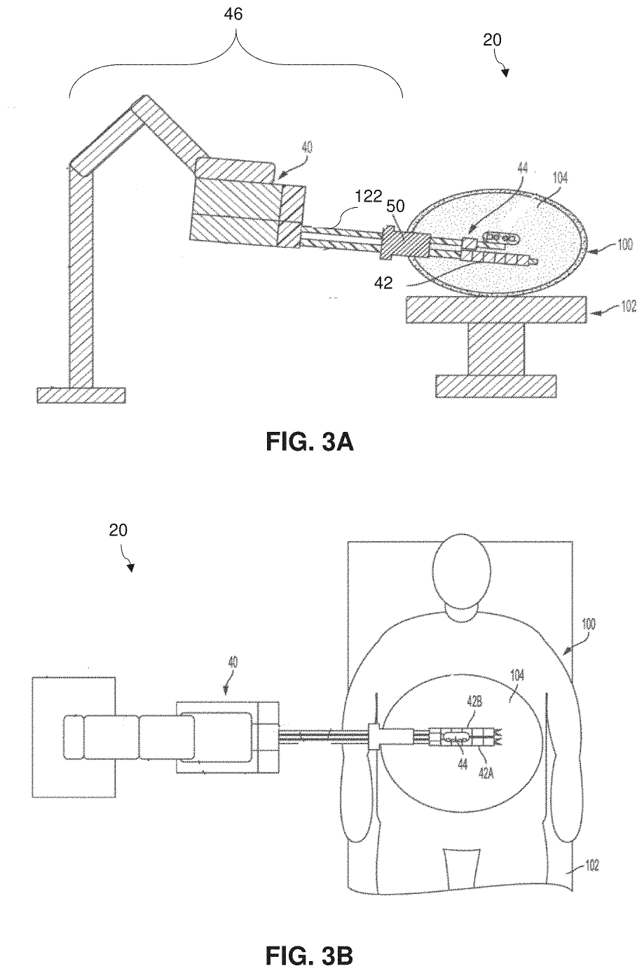

FIG. 3A schematically depicts a side view of the surgical robotic system 10 performing a surgery within an internal cavity 104 of a subject 100 in accordance with some embodiments and for some surgical procedures. FIG. 3B illustrates a perspective top view of the surgical robotic system 10 performing the surgery within the internal cavity 104 of the subject 100. The subject 100 (e.g., a patient) is placed on an operation table 102 (e.g., a surgical table 102). In some embodiments, and for some surgical procedures, an incision is made in the patient 100 to gain access to the internal cavity 104. The trocar 50 is then inserted into the patient 100 at a selected location to provide access to the internal cavity 104 or operation site. The RSS 46 can then be maneuvered into position over the patient 100 and the trocar 50. The robotic assembly 20 can be coupled to the motor unit 40 and at least a portion of the robotic assembly can be inserted into the trocar 50 and hence into the internal cavity 104 of the patient 100. For example, the camera assembly 44 and the robotic arm assembly 42 can be inserted individually and sequentially into the patient 100 through the trocar 50. Although the camera assembly and the robotic arm assembly may include some portions that remain external to the subject's body in use, references to insertion of the robotic arm assembly 42 and/or the camera assembly into an internal cavity of a subject and disposing the robotic arm assembly 42 and/or the camera assembly 44 in the internal cavity of the subject are referring to the portions of the robotic arm assembly 42 and the camera assembly 44 that are intended to be in the internal cavity of the subject during use. The sequential insertion method has the advantage of supporting smaller trocars and thus smaller incisions can be made in patient 100, thus reducing the trauma experienced by the patient 100. In some embodiments, the camera assembly 44 and the robotic arm assembly 42 can be inserted in any order or in a specific order. In some embodiments, the camera assembly 44 can be followed by a first robot arm of the robotic arm assembly 42 and then followed by a second robot arm of the robotic arm assembly 42 all of which can be inserted into the trocar 50 and hence into the internal cavity 104. Once inserted into the patient 100, the RSS 46 can move the robotic arm assembly 42 and the camera assembly 44 to an operation site manually or automatically controlled by the operator console 11 via different control modes (e.g., travel arm control mode, a camera control mode, a model manipulation control mode, or the like) as further described with respect to FIGS. 6-13.

Further disclosure control of movement of individual arms of a robotic arm assembly is provided in International Patent Application Publications WO 2022/094000 A1 and WO 2021/231402 A1, each of which is incorporated by reference herein in its entirety.

### Robotic Assembly Control

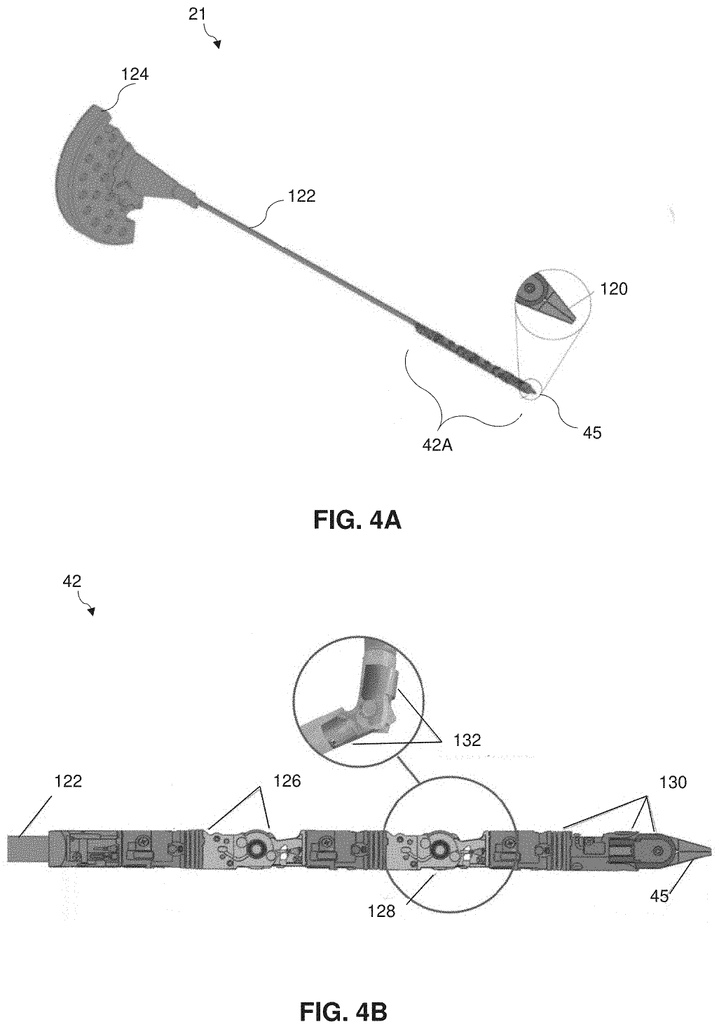

FIG. 4A is a perspective view of a robotic arm subassembly 21 in accordance with some embodiments. The robotic arm subassembly 21 includes a robotic arm 42A, the end-effector 45 having an instrument tip 120 (e.g., monopolar scissors, needle driver/holder, bipolar grasper, or any other appropriate tool), a shaft 122 supporting the robotic arm 42A. A distal end of the shaft 122 is coupled to the robotic arm 42A, and a proximal end of the shaft 122 is coupled to a housing 124 of the motor unit 40 (as shown in FIGS. 1 and 2A). At least a portion of the shaft 122 can be external to the internal cavity 104 (as shown in FIGS. 3A and 3B). At least a portion of the shaft 122 can be inserted into the internal cavity 10 (as shown in FIGS. 3A and 3B).

FIG. 4B is a side view of the robotic arm assembly 42. The robotic arm assembly 42 include a virtual shoulder 126, a virtual elbow 128 having capacitive proximity sensors 132, a virtual wrist 130, and the end-effector 45. The virtual shoulder 126, the virtual elbow 128, the virtual wrist 130 can include a series of hinge and rotary joints to provide each arm with positionable, seven degrees of freedom, along with one additional grasping degree of freedom for the end-effector 45.

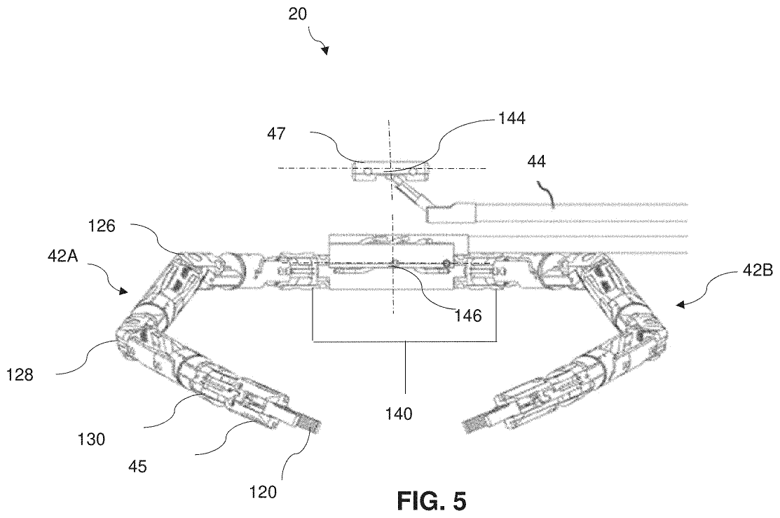

FIG. 5 illustrates a perspective front view an internal portion of the robotic assembly 20. The robotic assembly 20 includes a first robotic arm 42A and a second robotic arm 42B. The two robotic arms 42A and 42B can define a virtual chest 140 of the robotic assembly 20. The virtual chest 140 can be defined by a chest plane extending between a first pivot point 142A of a most proximal joint of the first robotic arm 42A, a second pivot point 142B of a most proximal joint of the second robotic arm 42B, and a camera imaging center point 144 of the camera(s) 47. A pivot center 146 of the virtual chest 140 lies midway along a line segment in the chest plane connecting the first pivot point 144 of the first robotic arm 42A and the second pivot point 142B of the second robotic arm. 42B.

FIG. 6 is a flowchart illustrating steps 200 for controlling the robotic assembly carried out by the surgical robotic system 100 of the present disclosure. Beginning in step 202, while at least a portion of the robotic assembly is disposed in an interior cavity of a subject, the surgical robotic system 10 receives a first control mode selection input from an operator, and changes a current control mode of the surgical robotic system 10 to a first control mode in response to the first control mode selection input. For example, as shown in FIGS. 3A and 3B, at least a portion of the robotic assembly 20, which may be referred to as an internal portion of the robotic assembly, is inserted in the interior cavity 104 of the subject 100 (e.g., a patient). While the internal portion of the robotic assembly 20 is disposed in the interior cavity 104 of the subject 100, the surgical robotic system 10 can receive a control mode selection input from the operator (e.g., a surgeon) via a control on the operator console 11, such as via one or both of the hand controllers 17, and/or one or more mode selection controllers 19 (e.g., foot pedals). For example, the operator can utilize a camera control foot pedal 19A to enter a camera control mode, and the operator can utilize a travel control foot pedal 19B to enter a travel arm control mode. In some embodiments, mode selection controls may also or alternatively be disposed on or in the hand controllers 17.

In step 204, while the surgical robotic system 10 is in the first control mode, the surgical robotic system 10 receives a first control input from hand controllers 17.

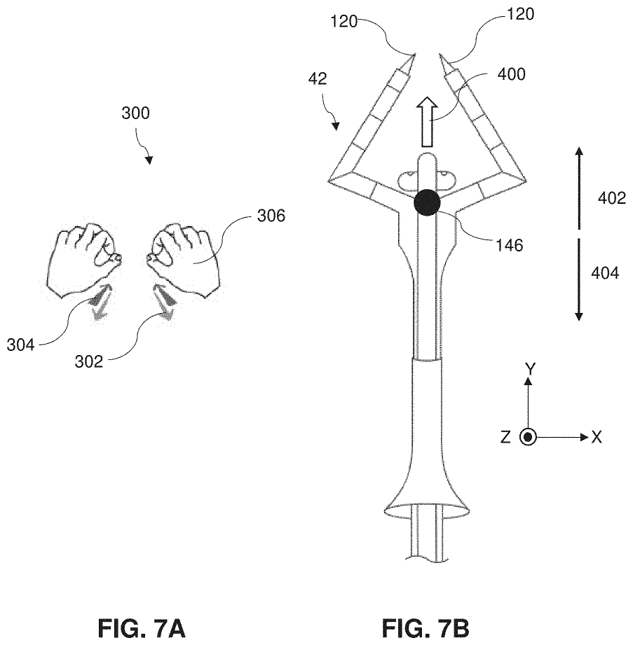

In a travel arm control mode, a control input can correspond to one of a plurality of gestural translation inputs (e.g., a pullback input, a push forward input, a horizontal input, a vertical input, a right yaw input, and/or a left yaw input) or one of a plurality of gestural rotation inputs (e.g., a pitch down input, a pitch up input, a clockwise roll input, and/or a counter-clockwise roll input). With respect to FIG. 5, if the control input corresponds to one of the plurality of gestural translation inputs, the surgical robotic system 10 moves at least the portion of the robotic arm assembly 42 to change the location of the virtual chest pivot center 146 while maintaining the stationary position of the instrument tips of the end effectors 45 in response to the control input. If control input corresponds to one of the plurality of gestural rotation inputs, the surgical robotic system 10 moves at least the portion of the robotic arm assembly 42 to change the orientation of the virtual chest 140 with respect to a current viewing direction of the camera(s) 47 while maintaining the stationary position of the instrument tips of the end effectors 45. Examples are further described with respect to FIGS. 7-12 and 16-19.

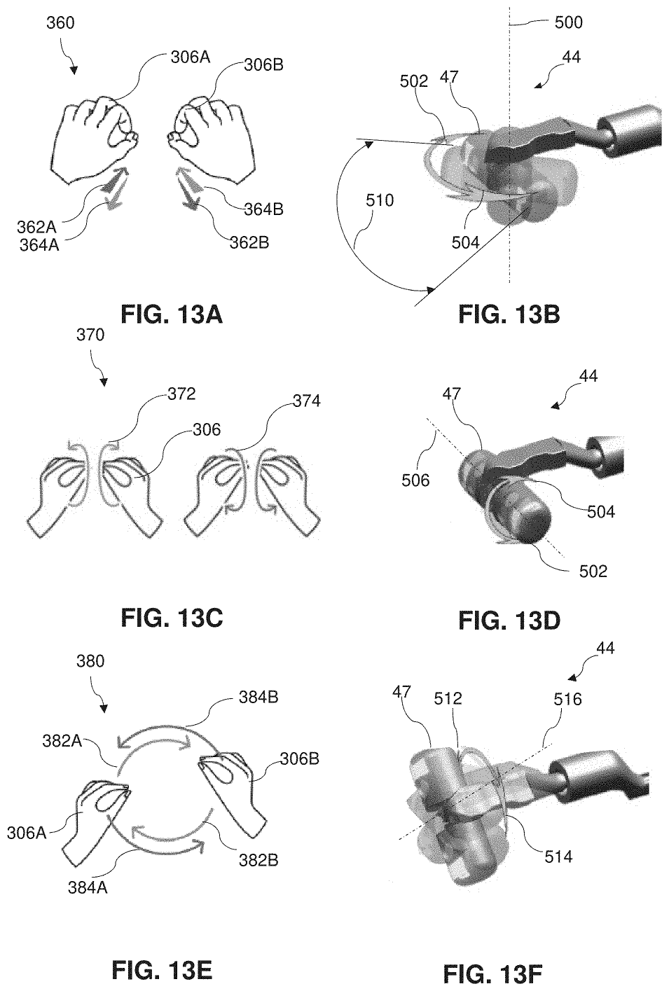

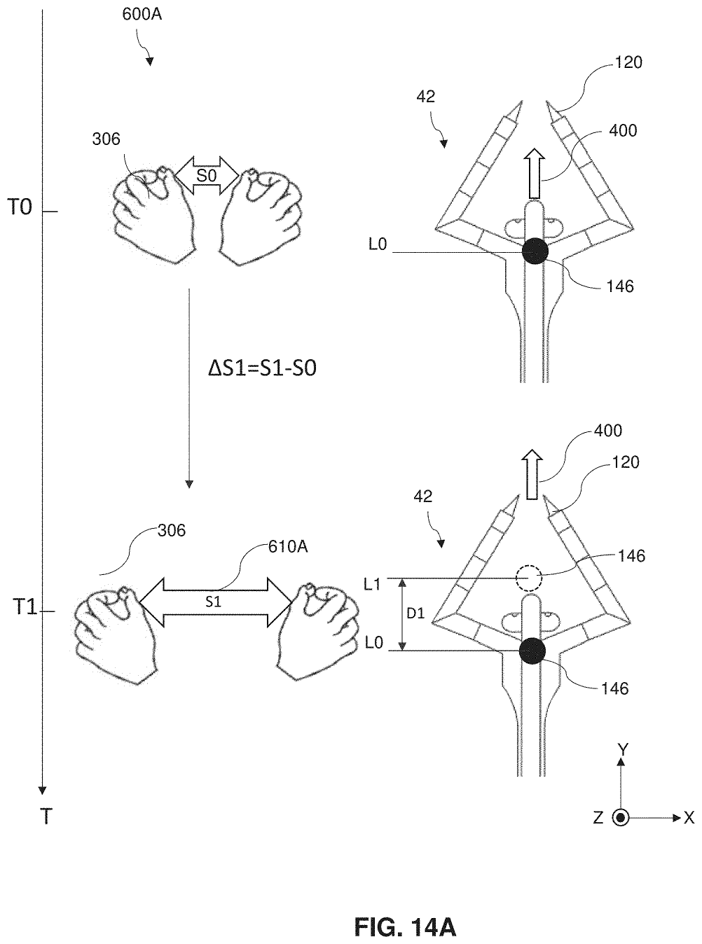

In a camera control mode, a control input can correspond to one of a plurality of gestural rotation inputs (e.g., a right yaw input, a left yaw input, a pitch down input, a pitch up input, a clockwise roll input, and/or a counter-clockwise roll input), as further described with respect to FIGS. 13A-13F.

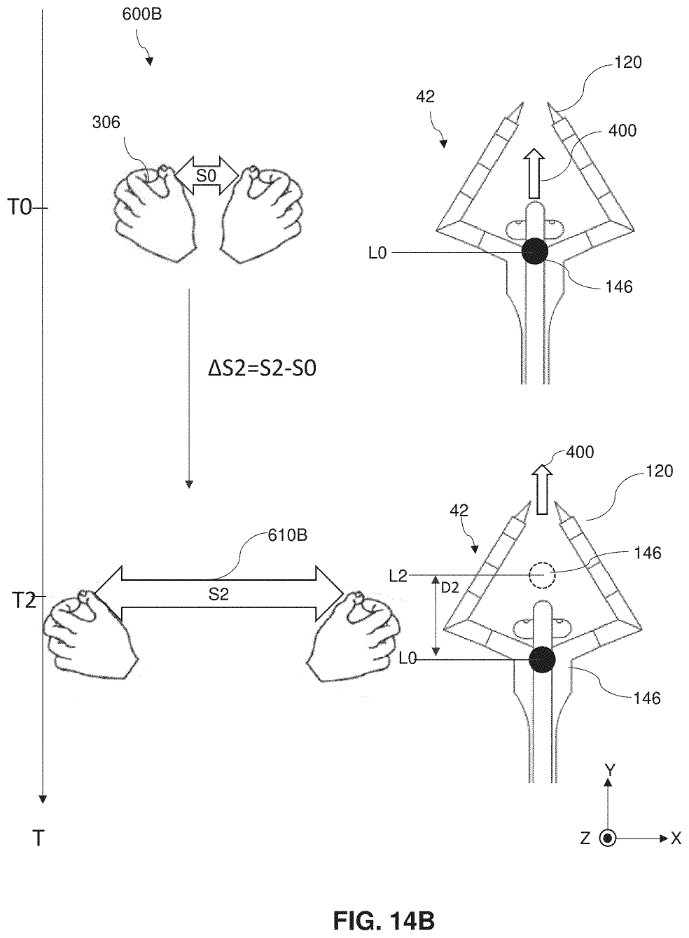

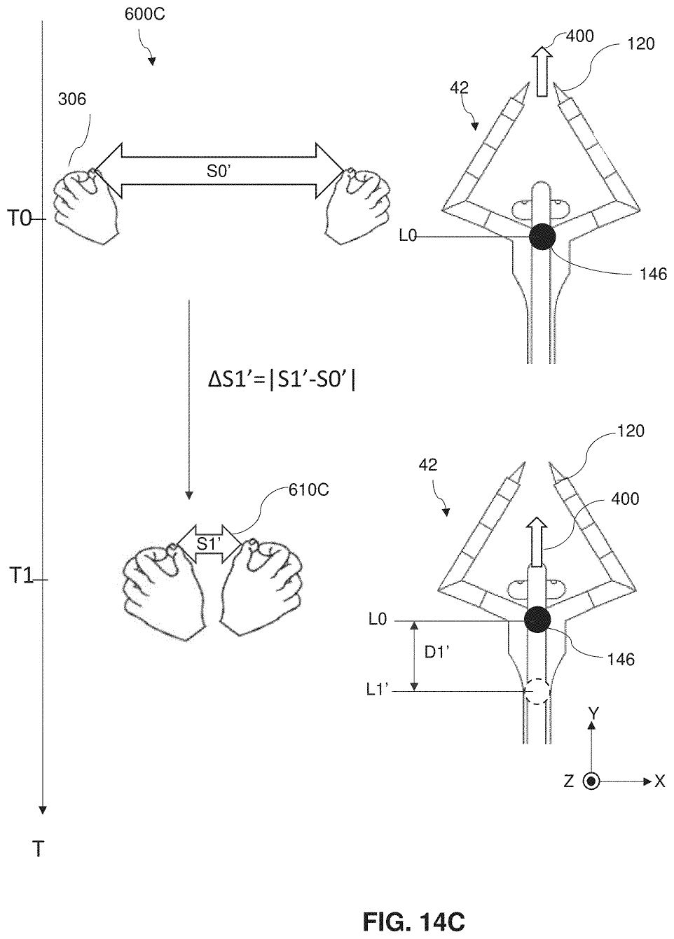

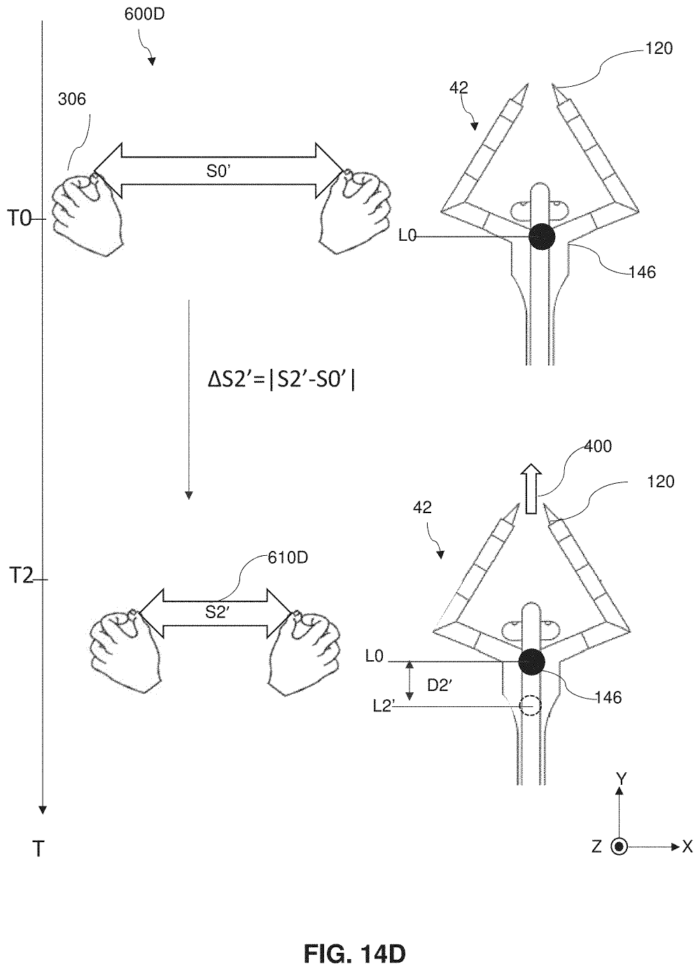

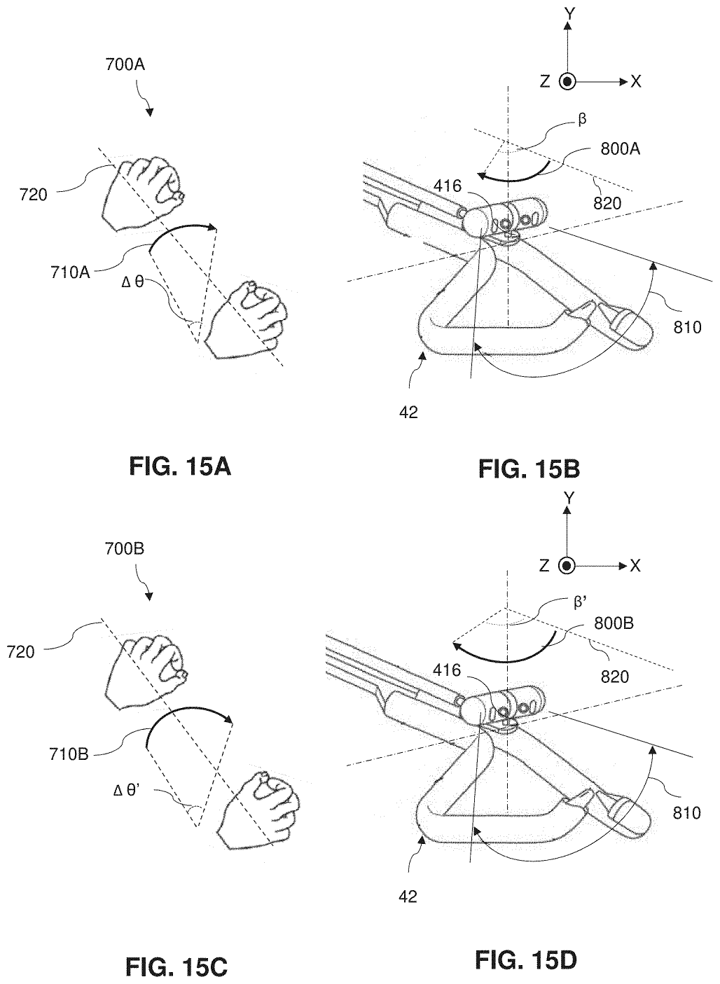

In a physical activity arm control mode, a control input can correspond to one of a plurality of different types of physical activity inputs (e.g., a zoom input and/or a wheel input), as further described with respect to FIGS. 14-15.

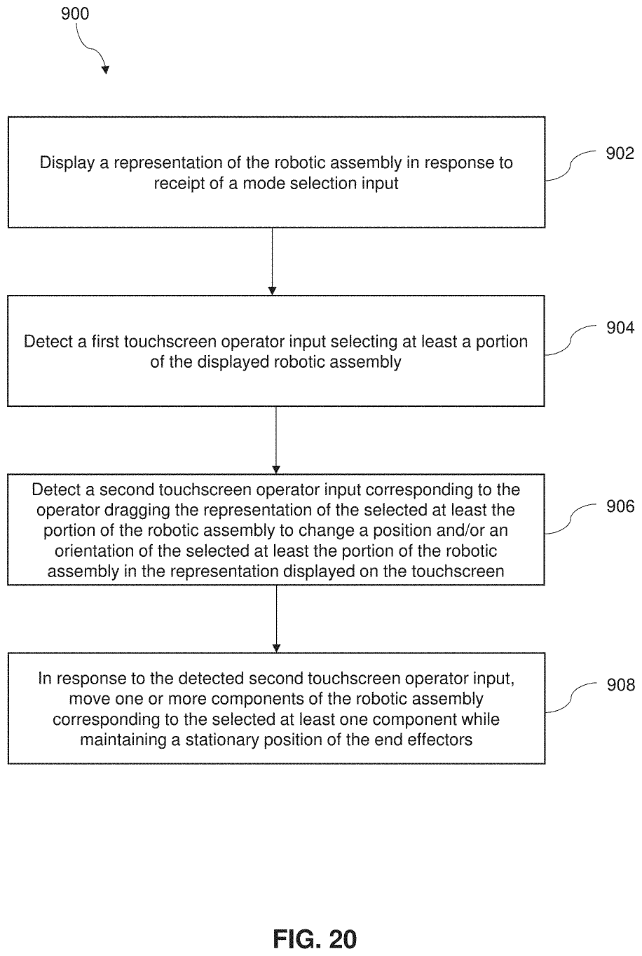

In a model manipulation control mode, a control input can correspond to a touchscreen operator input, as further described with respect to FIG. 20.

In step 206, in response to receiving the first control input, the surgical robotic system 10 changes a position and/or an orientation of: at least a portion of the camera assembly, of at least a portion of the robotic arm assembly, or both, while maintaining a stationary position of instrument tips of the end effectors disposed at distal ends of the robotic arms. The surgical robotic system 10 can include a plurality of control modes, such as travel arm control mode, a camera control mode, a physical activity arm control mode, a model manipulation control mode, or the like.

In a travel arm control mode, the surgical robotic system 10 can move at least a portion of the robotic arm assembly 42 to change a location of a virtual chest pivot center and/or an orientation of the virtual chest with respect to a current viewing direction of a camera, such as linearly repositioning the robotic arm assembly 42 and the camera assembly 44, and/or yawing, pitching, and/or rolling the robotic arm assembly 42 and the camera assembly 44. Examples are further described with respect to FIGS. 7-12, and 16-19. In a camera control mode, the surgical robotic system 10 can change an orientation and/or a position of at least one camera of the camera assembly 44 with respect to a current viewing direction (e.g., a reviewing direction of the camera) while keeping the robotic arm assembly 42 stationary, such as yawing, pitching, and/or rolling a field of view of the camera relative to the current viewing direction. Examples are described with respect to FIGS. 13A-13F.

In a physical activity arm control mode, one or more of: a magnitude of a translation of at least a portion of the robot arm assembly, a direction of the translation of at least the portion of the robotic arm assembly, a magnitude of a rotation of at least the portion of the robot arm assembly, and an axis of the rotation of at least the portion of the robotic arm assembly, depend, at least in part, on one or more of: a magnitude of the sensed movement of the hand controllers, a magnitude of a sensed change in separation between the hand controllers; a magnitude of a sensed change in lateral separation between the hand controller, a direction of a movement of the hand controllers, and a sensed change in orientation of a line connecting the hand controllers in the first control input. Examples are described with respect to FIGS. 14-15.

In a model manipulation control mode, the surgical robotic system 10 can move the robotic arm assembly 42 and/or the camera assembly 44 in response to a touchscreen operator input, as further descried with respect to FIG. 20.

### Gestural Arm Control Mode

FIG. 7A illustrates hand gestures 300 for a pullback input 302 and a push forward input 304 in a gestural arm control mode. FIG. 7B illustrates the movements of the robotic arm assembly 42 in response to the pullback input 302 and the push forward input 304. The pullback input 302 corresponds to the sensed movement of the hand controllers 17 (e.g., as shown in FIGS. 1 and 2B) corresponds to the operator's hands 306 moving back toward the operator's body. When in the gestural arm control mode, the surgical robotic system 10 moves at least the portion of the robotic arm assembly 42 to move the location of the virtual chest pivot center 146 forward 402 in the current viewing direction 400 in response to the pullback input 302 while maintaining the stationary position of the instrument tips of the end effectors 45. The push forward input 304 corresponds to the sensed movement of the hand controllers 17 (e.g., as shown in FIGS. 1 and 2B) corresponds to operator's hands 306 moving forward away from the operator's body. When in the gestural arm control mode, the surgical robotic system 10 moves at least the portion of the robotic arm assembly 42 to move the location of the virtual chest pivot center 146 back away 404 from the current viewing direction 400 in response to the push forward input 304 while maintaining the stationary position of the instrument tips 120 of the end effectors.

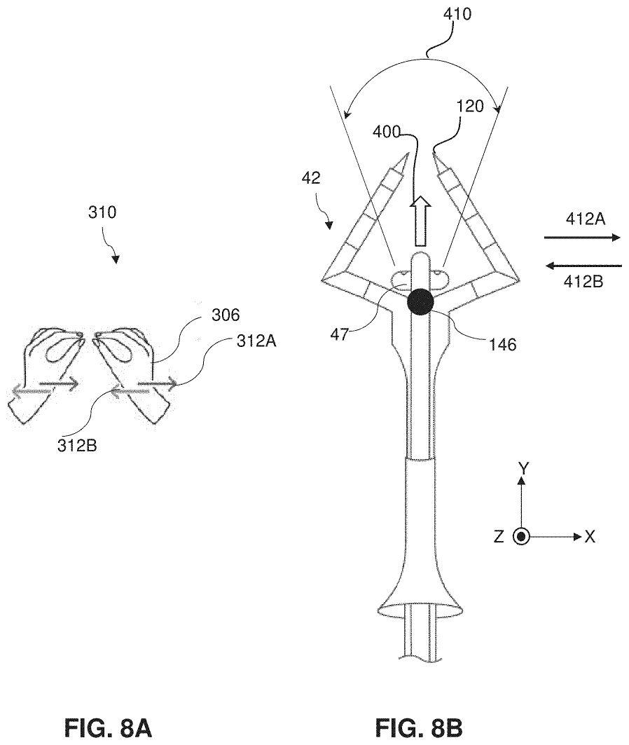

FIG. 8A illustrates hand gestures 310 for a horizontal input 312 in a gestural arm control mode. FIG. 8B illustrates the movements of the robotic arm assembly 42 in response to the horizontal input 312. The horizontal input 312 corresponds to the sensed movement of the hand controllers 17 (e.g., as shown in FIGS. 1 and 2B) corresponding to operator's hands 306 moving in a horizontal direction 412 with respect to the operator's body. When in the gestural arm control mode, the surgical robotic system 10 moves at least the portion of the robotic arm assembly 42 to move the location of the virtual chest pivot center 146 in a corresponding horizontal direction with respect to a current field of view 410 of the camera(s) 47 or a current image displayed. The corresponding horizontal direction is a horizontal direction to the left 412B or a horizontal direction to the right 412A with respect to the current viewing direction 400 or the current field of view 410 of the current image displayed in response to the horizontal input 312B or 312A, respectively, while maintaining the stationary position of the instrument tips 120 of the end effectors. It should be noted that the field of view may be wider or significantly wider than indicated by the lines depicted in the figures and marked 410. The lines depicted in the figures for the field of view 410 are merely for illustrative purposes and are not meant to reflect an actual field of view of the representative camera assembly.

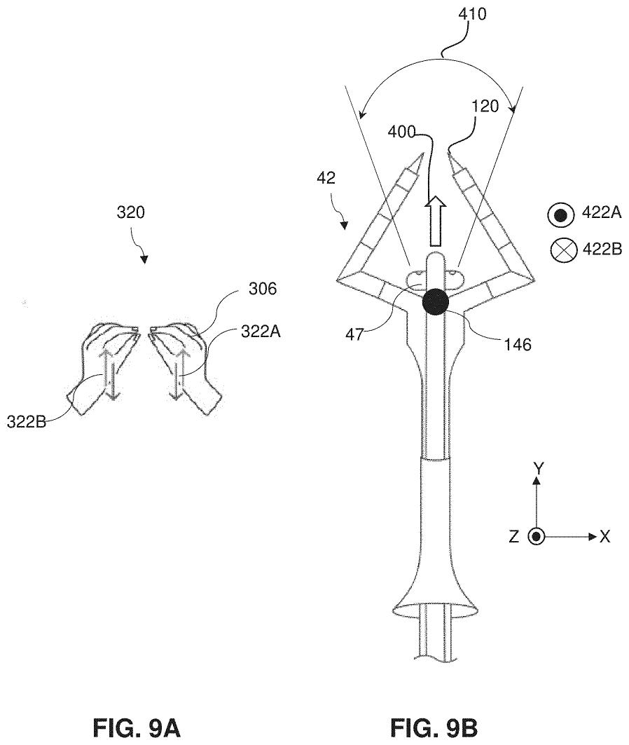

FIG. 9A illustrates hand gestures 320 for a vertical input 322 in a gestural arm control mode. FIG. 9B illustrates the movements of the robotic arm assembly 42 in response to the vertical input 322. The vertical input 422 corresponds to the sensed movement of the hand controllers 17 (e.g., as shown in FIGS. 1 and 2B) corresponding to operator's hands 306 moving in a vertical direction with respect to the operator's body. When in the gestural arm control mode, the surgical robotic system 10 moves at least the portion of the robotic arm assembly 42 to move the location of the virtual chest pivot center 416 in a corresponding vertical direction 422 with respect to the current viewing direction 400 or the current field of view 410, and wherein the corresponding vertical direction is a vertical up direction 422A or a vertical down direction 422B with respect to the current field of view 410 of the current image displayed in response to the vertical input 322A or 322B, respectively, while maintaining the stationary position of the instrument tips 120 of the end effectors.

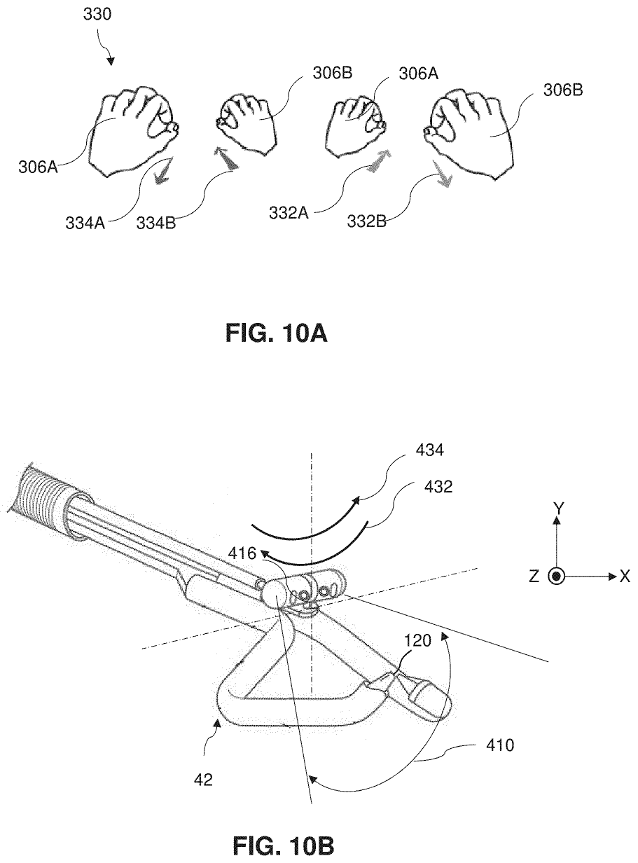

FIG. 10A illustrates hand gestures 330 for a right yaw input 332 and a left yaw input 334 in a gestural arm control mode. FIG. 10B illustrates the movements of the robotic arm assembly 42 in response to the right yaw input 332 and left yaw input 334. The right yaw input 332 corresponds to a sensed movement of a left hand controller corresponding to a left hand 306A of the operator moving forward away 332A from the operator's body and a sensed movement of a right hand controller corresponding to a right hand 306B of the operator moving back toward 332B the operator's body. When in the gestural arm control mode, the surgical robotic system 10 moves at least the portion of the robotic arm assembly 42 to yaw an orientation of the chest plane to the right 432 about the virtual chest pivot center 416 with respect to the current viewing direction or the current field of view 410 of the current image displayed in response to the right yaw input 332, while maintaining the stationary position of the instrument tips of the end effectors 45.

The left yaw input 334 corresponds to the sensed movement of the left hand controller corresponds to the operator's left hand 306A moving back toward 334A the operator's body and the sensed movement of the right hand controller corresponds to the operator's right hand 306B moving forward away 334B from the operator's body. When in the gestural arm control mode, the surgical robotic system 10 moves at least the portion of the robotic arm assembly 42 to yaw an orientation of the chest plane to the left 434 about the virtual chest pivot center 416 with respect to the current viewing direction 400 or the current field of view 410 in response to the left yaw input 334, while maintaining the stationary position of the instrument tips of the end effectors 45.

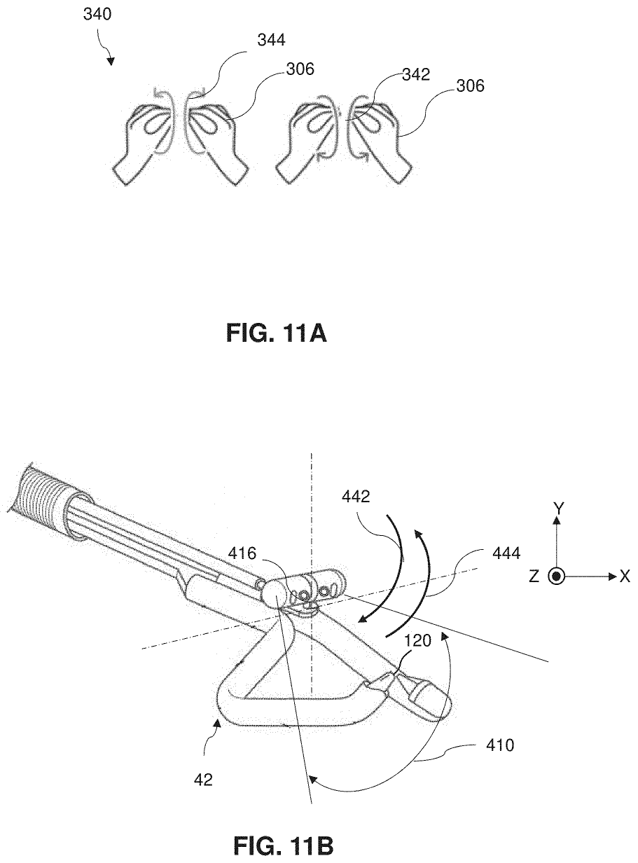

FIG. 11A illustrates hand gestures 340 for a pitch down input 342 and a pitch up input 344 in a gestural arm control mode. FIG. 11B illustrates the movements of the robotic arm assembly 42 in response to the pitch down input 342 and the pitch up input 342. The pitch down input 342 corresponds to the sensed movement of the hand controllers corresponding to the operator's hands tilting forward 342. When in the gestural arm control mode, the surgical robotic system 10 moves at least the portion of the robotic arm assembly 42 to pitch the orientation of the chest plane downward 442 about the virtual chest pivot center 416 with respect to the current viewing direction 400 or the current field of view 410 of the current image displayed in response to the pitch down input 342, while maintaining the stationary position of the instrument tips 120 of the end effectors.

The pitch up input 344 corresponds the sensed movement of the hand controllers and the sensed movement of the operator's hands 306 corresponds to the operator's hands tilting backward 344. When in the gestural arm control mode, the surgical robotic system moves at least the portion of the robotic arm assembly 42 to pitch the orientation of the chest plane upward 444 about the virtual chest pivot center 416 with respect to the current viewing direction 400 or the current field of view 410 in response to the pitch up input 344, while maintaining the stationary position of the instrument tips 120 of the end effectors.

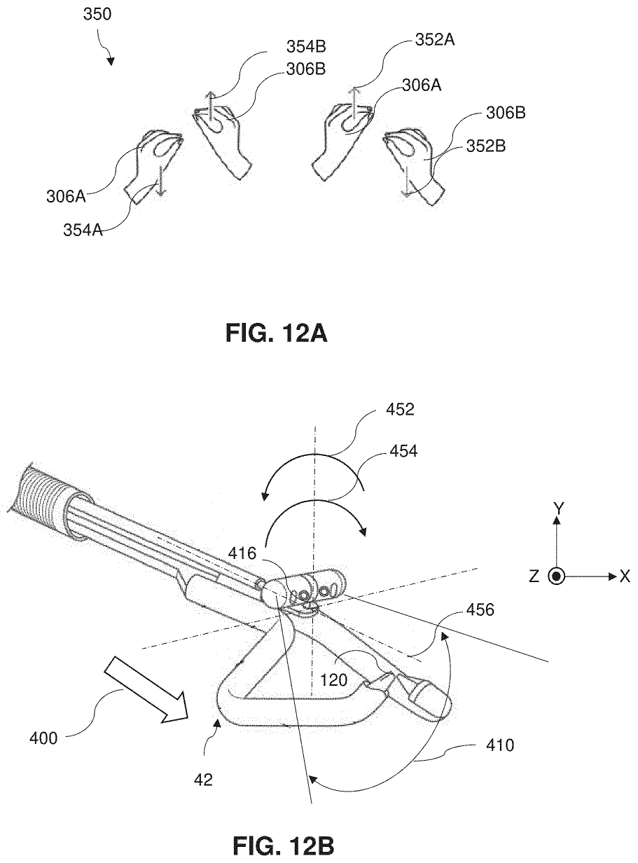

FIG. 12A illustrates hand gestures 350 for a clockwise roll input 352 and a counter-clockwise roll input 354 in a gestural arm control mode. FIG. 12B illustrates the movements of the robotic arm assembly 42 in response to the clockwise roll input 352 and the counter-clockwise roll input 354. The clockwise roll input 352 corresponds to a sensed movement of a left hand controller corresponding to a left hand 306A of the operator moving vertically up 352A and a sensed movement of the right hand controller corresponding to a right hand 306B of the operator moving vertically down 352B. When in the gestural arm control mode, the surgical robotic system 10 moves at least the portion of the robotic arm assembly 42 to rotate the robotic arm assembly 42 clockwise 452 about an axis 456 parallel to the current viewing direction 400 that passes through the virtual chest pivot center 416 with respect to the current viewing direction 400 or the current field of view 410 of the current image displayed in response to the clockwise roll input 352, while maintaining the stationary position of the instrument 120 tips of the end effectors.

The counter-clockwise roll input 354 corresponds to the sensed movement of the left hand controller corresponding to the operator's left hand 306A moving vertically down 354A and the sensed movement of the right hand controller corresponding to the operator's right hand 306B moving vertically up 354B. When in the gestural arm control mode, the surgical robotic system 10 moves at least the portion of the robotic arm assembly 42 to rotate the robotic arm assembly 42 counter-clockwise 454 about the axis 456 parallel to the current viewing direction 400 that passes through the virtual chest pivot center 416 with respect to the current field of view 410 in response to the counter-clockwise roll input 354, while maintaining the stationary position of the instrument tips of the end effectors 45.

### Gestural Camera Control Mode

FIG. 13A illustrates hand gestures 360 for a right yaw input 362 and a left yaw input 364 in a gestural camera control mode. FIG. 13B illustrates the movements of the camera assembly 44 in response to the right yaw input 362 and the left yaw input 364. The right yaw input 362 corresponds to a sensed movement of a left hand controller corresponding to a left hand 306A of the operator moving forward away 362A from the operator's body and a sensed movement of a right hand controller corresponding to a right hand 306B of the operator moving back toward 362B the operator's body. When in the gestural camera control mode, the surgical robotic system 10 moves at least a portion of the camera assembly 44 to yaw an orientation of a direction of view of the camera(s) 47 of the camera assembly 44 to the right 502 about a yaw rotation axis 500 of the camera assembly 44 with respect to a current field of view 510 of a current image displayed in response to the right yaw input 362.

The left yaw input 364 corresponds to the sensed movement of the operator's left hand corresponding to the operator's left hand 306A moving back toward 364A the operator's body and the sensed movement of the operator's right hand corresponding to the operator's right hand 306B forward away from the operator's body. When in the gestural camera control mode, the surgical robotic system 10 moves at least a portion of the camera assembly 44 to yaw an orientation of a direction of view of the camera(s) 47 to the left 504 about the yaw rotation axis 500 of the camera assembly with respect to the current field of view 510 of the current image displayed in response to the left yaw input 364.

FIG. 13C illustrates hand gestures 370 for a pitch down input 372 and a pitch up input 374 in a gestural camera control mode. FIG. 13D illustrates the movements of the camera assembly 44 in response to the pitch down input 372 and the pitch up input 374. The pitch down input 372 corresponds to the sensed movement of the hand controllers corresponding to the operator's hands tilting forward. When in the gestural camera control mode, the surgical robotic system 10 moves at least the portion of the camera assembly 44 to pitch an orientation of the direction of view of the camera(s) 47 downward 502 about a pitch axis 506 of the camera assembly 44 in response to the pitch down input 372.

The pitch up input 374 corresponds to the sensed movement of the hand controllers corresponding to the operator's hands tilting backward. When in the gestural camera control mode, the surgical robotic system 10 moves at least the portion of the cameras assembly 44 to pitch an orientation of the direction of view of the camera(s) 47 upward 504 about the pitch axis 506 of the camera assembly 44 in response to the pitch up input 374.

FIG. 13E illustrates hand gestures 380 for a clockwise roll input 382 and a counter-clockwise roll input 384 in a gestural camera control mode. FIG. 13F illustrates the movements of the camera assembly 44 in response to the clockwise roll input 382 and the counter-clockwise roll input 384. The clockwise roll input 382 corresponds to sensed movement of the hand controllers corresponding to the left hand 306A of the operator moving vertically up 382A and the right hand 306B of the operator moving vertically down 382B. When in the gestural camera control mode, the surgical robotic system 10 moves at least the portion of the camera assembly 44 to roll the camera 44 clockwise 512 about an axis 516 parallel to the current viewing direction in response to the clockwise roll input 382.

The counter-clockwise roll input 384 corresponds to the sensed movement of the hand controllers corresponding to the operator's left hand 306A moving vertically down 384A and the operator's right hand 306B moving vertically up 384B. When in the gestural camera control mode, the surgical robotic system 10 moves at least the portion of the camera assembly to roll the camera(s) 47 counter-clockwise 514 about the axis 516 parallel to the current viewing direction in response to the counter-clockwise roll input 384.

### Physical Activity Mode

FIG. 14A illustrates hand gestures 600A for a zoom input 610A in a physical activity control mode and the movements of the robotic arm assembly 42 in response to the zoom input 610A. At time T0, the hands 306 of the operator are located at initial location having an initial separation S0. At time T1, the hands 306 of the operator are laterally separated having a lateral separation S1. The zoom input 610A corresponds the sensed movement of the hand controllers 17 (as shown in FIGS. 1 and 2B) corresponding to a change (ΔS1=S1−S0) in lateral separation. The lateral separation increases from S0 at T0 to S1 at T1, and, in response, the surgical robotic system 10 moves at least the portion of the robotic arm assembly 42 to move the location L0 of the virtual chest pivot center 146 forward in the current viewing direction 400 to a location L1. The displacement between L0 and L1 is D1. The image displayed when the virtual chest pivot center 146 is at L0 may be zoomed in or appear zoomed in when the virtual chest pivot center 146 is moved from L0 to L1 due to the change in the position of virtual chest pivot center 146.

FIG. 14B illustrates hand gestures 600B for another zoom input 610B in the physical activity control mode and the movements of the robotic arm assembly 42 in response to the zoom input 610B. At time T2, the hands 306 of the operator are laterally separated having a lateral separation S2. The zoom input 610B corresponds the sensed movement of the hand controllers 17 (as shown in FIGS. 1 and 2B) corresponding to a change (ΔS2=S2−S0) in lateral separation. The lateral separation increases from S0 at T0 to S2 at T2, and, in response, the surgical robotic system 10 moves at least the portion of the robotic arm assembly 42 to move the location L0 of the virtual chest pivot center 146 forward in the current viewing direction 400 to a location L2. The displacement between L0 and L2 is D2. The image displayed when the virtual chest pivot center 146 is at L0 may be zoomed in or appear zoomed in when the virtual chest pivot center 146 is moved from L0 to L2 due to the change in the position of virtual chest pivot center 146.

Because ΔS2 is greater than ΔS1, D2 is greater than D1. A magnitude of a displacement of the virtual chest pivot center depends on a magnitude of the change in lateral separation in response to the zoom input 610A or 610B. The image displayed when the virtual chest pivot center 146 is at L2 is larger or may appear larger than the image displayed when the virtual chest pivot center 146 is at L1.

FIG. 14C illustrates hand gestures 600C for another zoom input 610C in a physical activity control mode and the movements of the robotic arm assembly 42 in response to the zoom input 610C. At time T0, the hands 306 of the operator are located at initial location having an initial separation S0′. At time T1, the hands 306 of the operator get closer having a lateral separation S1′. The zoom input 610C corresponds the sensed movement of the hand controllers 17 (as shown in FIGS. 1 and 2B) corresponding to a change (ΔS1′=|S1′−S0′|) in lateral separation. The lateral separation decreases from S0′ at T0 to S1′ at T1, and, in response, the surgical robotic system 10 moves at least the portion of the robotic arm assembly 42 to move the location L0 of the virtual chest pivot center 146 backward with respect to the current viewing direction 400 to a location L1′. The displacement between L0 and L1′ is D1′. The image displayed when the virtual chest pivot center 146 is at L0 may be zoomed in or may appear zoomed in when the virtual chest pivot center 146 is moved from L0 to L1′ due to the change in the position of virtual chest pivot center 146.

FIG. 14D illustrates hand gestures 600D for another zoom input 610D in the physical activity control mode and the movements of the robotic arm assembly 42 in response to the zoom input 610D. At time T2, the hands 306 of the operator get closer having a lateral separation S2′. The zoom input 610D corresponds the sensed movement of the hand controllers 17 (as shown in FIGS. 1 and 2B) corresponding to a change (ΔS2′=|S2′−S0′|) in lateral separation. The lateral separation decreases from S0′ at T0 to S2′ at T2, and, in response, the surgical robotic system 10 moves at least the portion of the robotic arm assembly 42 to move the location L0 of the virtual chest pivot center 146 backward with respect to the current viewing direction 400 to a location L2′. The displacement between L0 and L2′ is D2′. The image displayed when the virtual chest pivot center 146 is at L0 may be zoomed out or appear zoomed out when the virtual chest pivot center 146 is moved from L0 to L2′ due to the change in the position of virtual chest pivot center 146.

Because ΔS1′ is greater than ΔS2′, D1′ is greater than D2′. A magnitude of a displacement of the virtual chest pivot center depends on a magnitude of the change in lateral separation in response to the zoom input 610C or 610D. The image displayed when the virtual chest pivot center 146 is at L1′ is smaller or may appear smaller than the image displayed when the virtual chest pivot center 146 is at L2′.

FIGS. 15A and 15C illustrate hand gestures 700A, 700B for wheel inputs 710A and 710B corresponding to a clockwise rotation in a physical activity mode. FIGS. 15B and 15D illustrate the movements of the robotic arm assembly 42 in response to the wheel inputs 710A and 710B.

The wheel input 710A corresponds to an angular change Δθ in an orientation of a line 720 connecting the hand controllers 17 (as shown in FIGS. 1 and 2B) in a vertical plane. When the change Δθ in orientation corresponds to clockwise rotation, the surgical robotic system 100 moves at least the portion of the robotic arm assembly 42 to rotate the orientation of the virtual chest to the right 800A having an angle β with respect to a current field of view 810 of a current image displayed and/or a viewing direction 820. Similarly, the wheel input 710B corresponds to an angular change Δθ′ in an orientation of the line 720 connecting the hand controllers 17 (as shown in FIGS. 1 and 2B) in the vertical plane. When the change Δθ′ in orientation corresponds to a clockwise rotation, the surgical robotic system 100 moves at least the portion of the robotic arm assembly 42 to rotate the orientation of the virtual chest to the right 800B having an angle β′ with respect to the current field of view 810 of the current image displayed and/or the viewing direction 820. A magnitude of the angular rotation of the virtual chest depends on a magnitude of the angular change in the orientation of the line in response to the wheel input 710A. For example, the angular change AO of the wheel input 710A is less than the angular change Δθ′ of the wheel input 710B. In response to the wheel input 710B, the at least the portion of the robotic arm assembly 42 rotates in a greater angle β′ than the angle β caused by the wheel input 710A.

Similar to the clockwise rotation, when a wheel input has a change in orientation corresponding to a counter-clockwise rotation (not shown), the surgical robotic system 10 moves at least the portion of the robotic arm assembly 42 to rotate the orientation of the virtual chest to the left with respect to the current field of view with the magnitude of the angular rotation of the virtual chest depending on the magnitude of the angular change in the orientation of the line in response to the wheel input.

### Examples of Changes in Orientation in Robotic Arms

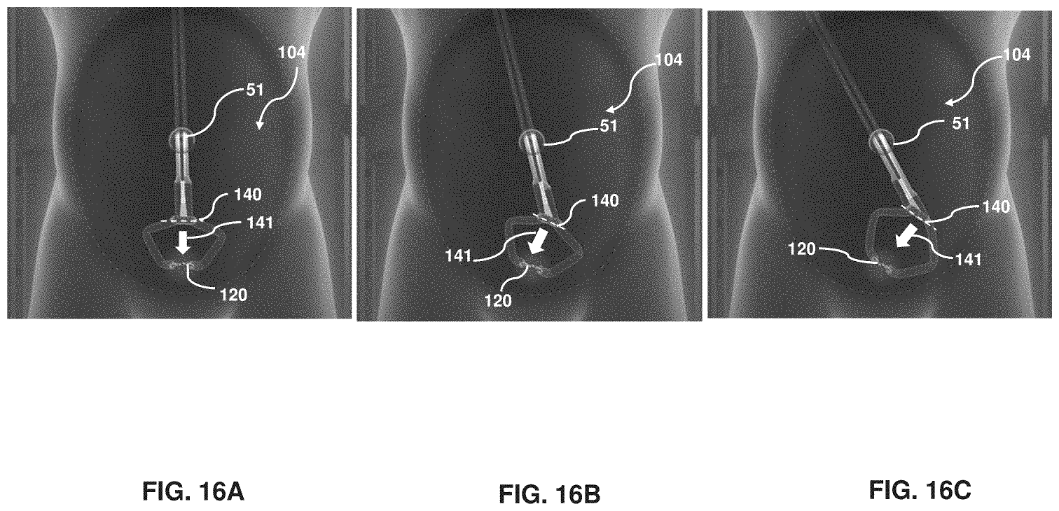

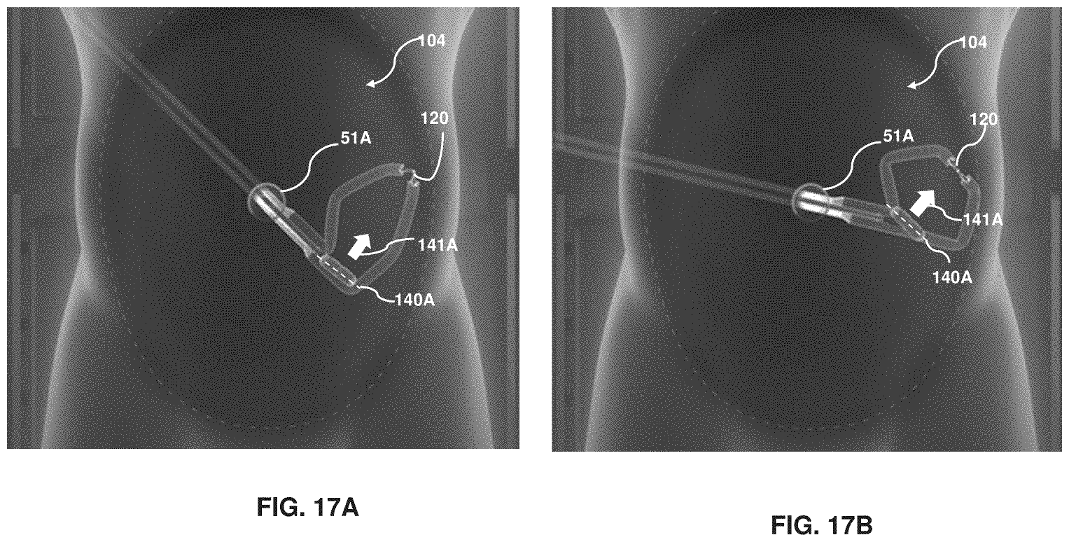

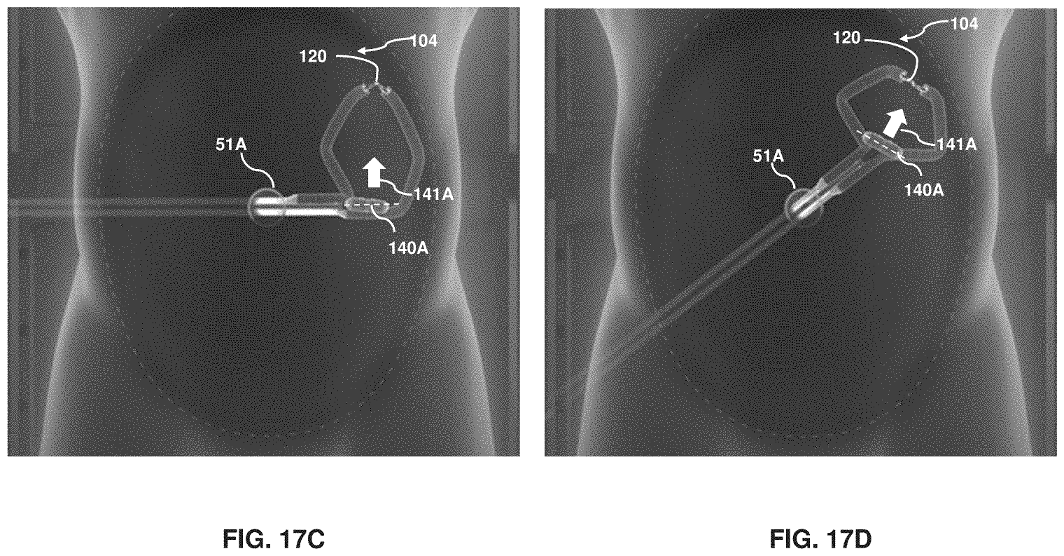

Each set of FIGS. 16A-16C, 17A-D, 18A-18C, and 19A-19B illustrate different orientations 141 and positions of the virtual chest 140 of the robotic arm assembly that can achieved while maintaining a stationary position of end effectors 120 and a stationary position of trocar pivot center 51.

### Model Manipulation Control Mode

FIG. 20 is a flowchart illustrating steps 900 performing a model manipulation control mode carried out by the surgical robotic system 10 of the present disclosure. In step 902, the surgical robotic system 10 displays a representation of the robotic assembly 20 in response to receipt of a mode selection input. For example, with respect to FIGS. 1, 2A, 2B, 3A and 3B, after insertion of the robotic arm assembly 42 and the camera assembly 44 into the interior cavity 104 of the subject 100, the surgical robotic system 10 can receive a mode selection input via the mode selection controller 19 indicating that the operator selects a model manipulation control mode. The surgical robotic system 10 can change a current control mode to the model manipulation control mode. The surgical robotic system 10 displays a representation of the robotic assembly 20 via the display unit 12 having a touchscreen. In some embodiments, the surgical robotic system 10 displays a representation of the robotic arm assembly 42 and the camera assembly 44 with respect to the X-Y and X-Z planes. In some embodiments, the surgical robotic system 10 can display a 3D model representing the robotic arm assembly 42 and the camera assembly 44.

In step 904, the surgical robotic system 10 detects a first touchscreen operator input selecting at least a portion of the displayed robotic assembly. For example, with respect to FIG. 5, the operator may select one or more of: the virtual shoulder 126, the virtual elbow 128, the virtual wrist 130, and the virtual chest 140.

In step 906, the surgical robotic system 10 detects a second touchscreen operator input corresponding to the operator dragging the representation of the selected at least the portion of the robotic assembly to change a position and/or an orientation of the selected at least the portion of the robotic assembly in the representation displayed on the touchscreen. For example, with respect to FIGS. 7-15, instead of using the hand gestures for control inputs, the operator may touch the touchscreen to select a representation of the virtual chest 140 or other regions shown in FIG. 5 and drag the representation of selected virtual chest 140 to a different location in the representation displayed on the touchscreen.

In step 908, in response to the detected second touchscreen operator input, the surgical robotic system 10 moves one or more components of the robotic assembly corresponding to the selected at least one component while maintaining a stationary position of the instrument tips of the end effectors. For example, the surgical robotic system 10 moves the robotic arm assembly 42 and the camera assembly 44 to a location in the internal cavity corresponding to the location in the representation displayed on the touchscreen.

While preferred embodiments of the present disclosure ion have been shown and described herein, it will be obvious to those skilled in the art that such embodiments are provided by way of example only. Numerous variations, changes, and substitutions will now occur to those skilled in the art without departing from the invention. It may be understood that various alternatives to the embodiments of the invention described herein may be employed in practicing the invention. It is intended that the following claims define the scope of the invention and that methods and structures within the scope of these claims and their equivalents be covered thereby.

1. A method for controlling a robotic assembly of a surgical robotic system, the surgical robotic system comprising an image display, hand controllers configured to sense a movement of an operator's hands, the robotic assembly including a camera assembly and a robotic arm assembly including a first robotic arm and a second robotic arm, the method comprising:
while at least a portion of the robotic assembly is disposed in an interior cavity of a subject, receiving a first control mode selection input from the operator and changing a current control mode of the surgical robotic system to a first control mode in response to the first control mode selection input;
while the surgical robotic system is in the first control mode, receiving a first control input from hand controllers; and
in response to receiving the first control input, the surgical robotic system changing a position and/or an orientation of: at least a portion of the camera assembly, at least a portion of the robotic arm assembly, or both, while maintaining a stationary position of instrument tips of end effectors disposed at distal ends of the robotic arms.

2. The method of claim 1, wherein the first robotic arm and the second robotic arm define a virtual chest of the robotic assembly, the virtual chest defined by a chest plane extending between a first pivot point of a most proximal joint of the first robotic arm, a second pivot point of a most proximal joint of the second robotic arm, and a camera imaging center point of the camera assembly; and
wherein a pivot center of the virtual chest lies midway along a line segment in the chest plane connecting the first pivot point of the first robotic arm and the second pivot point of the second robotic arm.

3. The method of claim 2, wherein the first control mode is a travel arm control mode or a camera control mode; and
where the first control mode is a camera control mode, in response to receiving the first control input, the surgical robotic system changing an orientation and/or a positon of at least one camera of the camera assembly with respect to the current viewing direction while keeping the robotic arm assembly stationary; and
where the first control mode is a travel arm control mode, in response to receiving the first control input, the surgical robotic system moving at least a portion of the robotic arm assembly to change a location of the virtual chest pivot center and/or an orientation the virtual chest with respect to the current viewing direction.

4. The method of claim 2, wherein the first control mode is a travel gestural arm control mode; and
wherein the first control input corresponds to one of a plurality of gestural translation inputs or one of a plurality of gestural rotation inputs;
where the first control input corresponds to one of the plurality of gestural translation inputs, the surgical robotic system moves at least the portion of the robotic arm assembly to change the location of the virtual chest pivot center while maintaining the stationary position of the instrument tips of the end effectors in response to the first control input; and
where the first control input corresponds to one of the plurality of gestural rotation inputs, the surgical robotic system moves at least the portion of the robotic arm assembly to change the orientation of the virtual chest with respect to the current viewing direction while maintaining the stationary position of the instrument tips of the end effectors.

5. The method of claim 4, wherein the plurality of gestural translation inputs comprises:
a pullback input in which the sensed movement of the hand controllers corresponds to the operator's hands moving back toward the operator's body, and where, when in the gestural arm control mode, the surgical robotic system moves at least the portion of the robotic arm assembly to move the location of the virtual chest pivot center forward in the current viewing direction in response to the pullback input; and
a push forward input in which the sensed movement of the hand controllers corresponds to operator's hands moving forward away from the operator's body, and where, when in the gestural arm control mode, the surgical robotic system moves at least the portion of the robotic arm assembly to move the location of the virtual chest pivot center back away from the current viewing direction in response to the push forward input.

6. The method of claim 4, wherein the plurality of gestural translation inputs comprises:
a horizontal input, in which the sensed movement of the hand controllers corresponds to operator's hands moving in a horizontal direction with respect to the operator's body, and where, when in the gestural arm control mode, the surgical robotic system moves at least the portion of the robotic arm assembly to move the location of the virtual chest pivot center in a corresponding horizontal direction with respect to a current field of view of a current image displayed, and wherein the corresponding horizontal direction is a horizontal direction to the left or a horizontal direction to the right with respect to the current field of view of the current image displayed in response to the horizontal input.

7. The method of claim 4, wherein the plurality of gestural translation inputs comprises:
a vertical input, in which the sensed movement of the hand controllers corresponds to operator's hands moving in a vertical direction with respect to the operator's body, and where, when in the gestural arm control mode, the surgical robotic system moves at least the portion of the robotic arm assembly to move the location of the virtual chest pivot center in a corresponding vertical direction with respect to a current field of view of a current image displayed, and wherein the corresponding vertical direction is a vertical up direction or a vertical down direction with respect to the current field of view of the current image displayed in response to the vertical input.

8. The method of claim 4, wherein the plurality of gestural rotation inputs comprises:
a right yaw input, in which a sensed movement of a left hand controller corresponds to a left hand of the operator moving forward away from the operator's body and a sensed movement of a right hand controller corresponds to a right hand of the operator moving back toward the operator's body, and where, when in the gestural arm control mode, the surgical robotic system moves at least the portion of the robotic arm assembly to yaw an orientation of the chest plane to the right about the virtual chest pivot center with respect to a current field of view of a current image displayed in response to the right yaw input; and
a left yaw input, in which the sensed movement of the left hand controller corresponds to the operator's left hand moving back toward the operator's body and the sensed movement of the right hand controller corresponds to the operator's right hand moving forward away from the operator's body, and where, when in the gestural arm control mode, the surgical robotic system moves at least the portion of the robotic arm assembly to yaw an orientation of the chest plane to the left about the virtual chest pivot center with respect to the current field of view in response to the left yaw input.

9. The method of claim 4, wherein the plurality of gestural rotation inputs comprises:
a pitch down input, in which the sensed movement of the hand controllers corresponds to the operator's hands tilting forward, and where, when in the gestural arm control mode, the surgical robotic system moves at least the portion of the robotic arm assembly to pitch the orientation of the chest plane downward about the virtual chest pivot center with respect to a current field of view of a current image displayed in response to the pitch down input; and
a pitch up input in which the sensed movement of the hand controllers and the sensed movement of the operator's hands corresponds to the operator's hands tilting backward, and where, when in the gestural arm control mode, the surgical robotic system moves at least the portion of the robotic arm assembly to pitch the orientation of the chest plane upward about the virtual chest pivot center with respect to the current field of view in response to the pitch up input.

10. The method of claim 4, wherein the plurality of gestural rotation inputs comprises:
a clockwise roll input, in which a sensed movement of a left hand controller corresponds to a left hand of the operator moving vertically up and a sensed movement of the right hand controller corresponds to a right hand of the operator moving vertically down, and where, when in the gestural arm control mode, the surgical robotic system moves at least the portion of the robotic arm assembly to rotate the robotic arm assembly clockwise about an axis parallel to the current viewing direction that passes through the virtual chest pivot center with respect to a current field of view of a current image displayed in response to the clockwise roll input; and
a counter-clockwise roll input, in which the sensed movement of the left hand controller corresponds to the operator's left hand moving vertically down and the sensed movement of the right hand controller corresponds to the operator's right hand moving vertically up, and where, when in the gestural arm control mode, the surgical robotic system moves at least the portion of the robotic arm assembly to rotate the robotic arm assembly counter-clockwise about an axis parallel to the current viewing direction that passes through the virtual chest pivot center with respect to the current field of view in response to the counter-clockwise roll input.

11. The method of claim 2, wherein the first control mode is a physical activity arm control mode, in which one or more of: a magnitude of a translation of at least a portion of the robot arm assembly, a direction of the translation of at least the portion of the robotic arm assembly, a magnitude of a rotation of at least the portion of the robot arm assembly, and an axis of the rotation of at least the portion of the robotic arm assembly, depend, at least in part, on one or more of: a magnitude of the sensed movement of the hand controllers; a magnitude of a sensed change in separation between the hand controllers; a magnitude of a sensed change in lateral separation between the hand controllers; a direction of a movement of the hand controllers, and a sensed change in orientation of a line connecting the hand controllers in the first control input; and
wherein the first control input corresponds to one of a plurality of different types of physical activity control inputs.

12. The method of claim 11, wherein the plurality of different types of physical activity control inputs comprises:
a zoom input, in which the sensed movement hand controllers corresponds to a change in lateral separation between the hand controllers,
where the lateral separation between the hand controllers increases, the surgical robotic system moves at least the portion of the robotic arm assembly to move the location of the virtual chest pivot center forward in the current viewing direction with a magnitude of a displacement of the virtual chest pivot center depending on a magnitude of the change in lateral separation in response to the zoom input, and
where the lateral separation between the hand controllers decreases, the surgical robotic system moves at least the portion of the robotic arm assembly to move the location of the virtual chest pivot center backward with respect to the current viewing direction with the magnitude of a displacement of the virtual chest pivot depending on the magnitude of the change in lateral separation in response to the zoom input.

13. The method of claim 11, wherein the plurality of different types of physical activity control inputs comprises:
a wheel input, in which the sensed movement of the hand controllers correspond to an angular change in an orientation of a line connecting the hand controllers in a vertical plane,
where the change in orientation corresponds to clockwise rotation, the surgical robotic system moves at least the portion of the robotic arm assembly to rotate the orientation of the virtual chest to the right with respect to a current field of view of a current image displayed with a magnitude of the angular rotation of the virtual chest depending on a magnitude of the angular change in the orientation of the line in response to the wheel input, and
where the change in orientation corresponds to a counter-clockwise rotation, the surgical robotic system moves at least the portion of the robotic arm assembly to rotate the orientation of the virtual chest to the left with respect to the current field of view with the magnitude of the angular rotation of the virtual chest depending on the magnitude of the angular change in the orientation of the line in response to the wheel input.

14-17. (canceled)

18. The method of claim 1, wherein the first control mode selection input is received via an input mechanism on one or both of the hand controllers.

19. The method of claim 1, wherein the first control mode selection input is received via a control on an operator console.

20. The method of claim 19, wherein the first control mode selection input is received via a foot pedal.

21. The method of claim 1, further comprising receiving a second mode selection input and changing a current control mode of the surgical robotic system to a second control mode.

22. The method of claim 21, wherein the first control mode is a travel arm control mode and the second control mode is a camera control mode.

23. The method of claim 21, wherein the first control mode is a travel arm control mode and the second control mode is a different travel arm control mode.

24. (canceled)

25. The method of claim 21, wherein when in the second control mode, the surgical robotic system maintains the robotic assembly in a stationary position and a static configuration regardless of the hand controller movement.

26-33. (canceled)

34. A surgical robotic system for performing a surgery within an internal cavity of a subject, the surgical robotic system comprising:
hand controllers operated to manipulate the surgical robotic system;
a computing unit configured to:
receive operator generated movement data from the hand controllers and to generate control signals in response based on a current control mode of the surgical robotic system; and
receive control mode selection input to change a current control mode of the surgical robotic system to a selected one of a plurality of control modes of the surgical robotic system in response;
a camera assembly; and
a robotic arm assembly configured to be inserted into the internal cavity during use, the robotic arm assembly including:
a first robotic arm including a first end effector disposed at a distal end of the first robotic arm; and
a second robotic arm including a second end effector disposed at a distal end of the second robotic arm; and
an image display for outputting an image from the camera assembly.

35-66. (canceled)
